(19) United States
(12) Patent Application Publication
(10) Pub. No.: US 2024/0011062 A1
(43) Pub. Date: Jan. 11, 2024

(54) METHOD TO PRODUCE A PURIFIED MIXTURE OF DIFFERENT OLIGOSACCHARIDES PRODUCED BY CELL CULTIVATION OR MICROBIAL FERMENTATION

(71) Applicant: INBIOSE N.V., Zwijnaarde (BE)

(72) Inventors: Joeri Beauprez, Zwijnaarde (BE); Maarten Delforche, Zwijnaarde (BE); Gaspard Lequeux, Zwijnaarde (BE); Wim Soetaert, Zwijnaarde (BE); Thomas Verbrugghen, Zwijnaarde (BE)

(21) Appl. No.: 18/041,019

(22) PCT Filed: Aug. 10, 2021
(86) PCT No.: PCT/EP2021/072272

§ 371 Date: Feb. 8, 2023

(30) Foreign Application Priority Data
Aug. 10, 2020 (EP) 20190209.5

(51) Int. Cl. C12P 19/04; B01D 15/36; C07H 1/08

(52) U.S. Cl. CPC C12P 19/04; B01D 15/361; C07H 1/08

(57) ABSTRACT

This disclosure is in the technical field of cell cultivation or fermentation for the production of oligosaccharides. This disclosure discloses a method to produce a purified mixture of different oligosaccharides produced by a cell cultivation or microbial fermentation.

### CROSS-REFERENCE TO RELATED APPLICATIONS

This application is a national phase entry under 35 U.S.C. § 371 of International Patent Application PCT/EP2021/072272, filed Aug. 10, 2021, designating the United States of America and published as International Patent Publication WO 2022/034078 A1 on Feb. 17, 2022, which claims the benefit under Article 8 of the Patent Cooperation Treaty to European Patent Application Serial No. 20190209.5, filed Aug. 10, 2020.

### TECHNICAL FIELD

This disclosure is in the technical field of cell cultivation or fermentation for the production of oligosaccharides. This disclosure discloses a method to produce a purified mixture of different oligosaccharides produced by a cell cultivation or microbial fermentation.

### BACKGROUND

Oligosaccharides, often present as glyco-conjugated forms to proteins and lipids, are involved in many vital phenomena such as differentiation, development and biological recognition processes related to the development and progress of fertilization, embryogenesis, inflammation, metastasis and host pathogen adhesion. Oligosaccharides can also be present as unconjugated glycans in body fluids and human milk wherein they also modulate important developmental and immunological processes (Bode, Early Hum. Dev. 1-4 (2015); Reily et al., Nat. Rev. Nephrol. 15, 346-366 (2019); Varki, Glycobiology 27, 3-49 (2017)). For example, several oligosaccharides have proven to act as decoys to reduce the risk of infections by bacterial and viral pathogens adhering to mammal cells by binding to these cells' surface glycoproteins. Nowadays, oligosaccharides are produced on an industrial scale either chemically, by chemo-enzymatic synthesis or by cultivation or fermentation of (metabolically engineered) cells or micro-organisms. After production, the oligosaccharide preferably is purified to be added in the respective application.

To take advantage of the positive effects of specific oligosaccharides, individual oligosaccharides are being added to nutritional compositions, cosmetics, pharmaceutical compositions and plant protection products. In some instances, supplementing with a combination of different oligosaccharides is more convenient, as such compositions e.g., more closely resemble the natural source of the oligosaccharides in case the oligosaccharide mixture is a mixture of mammalian milk oligosaccharides. In other cases a mix of specific oligosaccharides is produced more efficiently in a simpler manner by producing the mixture of oligosaccharides in one cultivation or fermentation and purifying the mixture of oligosaccharides all together from the biomass, medium components and contaminants, without separating the different oligosaccharides from each other.

### BRIEF SUMMARY

Provided are a purified mixture of different oligosaccharides, preferably in a solid form, and associated process, wherein the process is applicable in industrial scale production and does not involve separation in the purification of the different oligosaccharides with subsequent admixing of the different oligosaccharides in the final solution or powder.

Also provided is a method to produce a purified mixture of different oligosaccharides produced by a cell cultivation or microbial fermentation.

In a first aspect, a method to produce a purified mixture of different oligosaccharides produced by at least one cell, preferably a cell of a micro-organism, which synthesizes the mixture of different oligosaccharides, is provided.

In a second aspect, a method to produce a spray dried powder essentially comprising or containing a mixture of structurally distinct oligosaccharides is provided.

In a third aspect, a method to produce a syrup of a mixture of different oligosaccharides is provided with a Brix between 8% and 75% is provided.

In a fourth aspect, a spray dried powder essentially comprising or containing a mixture of structurally distinct oligosaccharides is provided, preferably for the production of a nutritional composition, a dietary supplement, a pharmaceutical ingredient, and/or a cosmetics ingredient.

In a fifth aspect, a nutritional composition comprising a spray-dried powder that essentially comprises or contains a mixture of structurally distinct oligosaccharides is provided.

### Definitions

The words used in this specification to describe this disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of this disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, purification steps are performed according to the manufacturer's specifications.

In the specification, there have been disclosed embodiments of this disclosure, and although specific terms are employed, the terms are used in a descriptive sense only and not for purposes of limitation, the scope of this disclosure being set forth in the following claims. It must be understood that the illustrated embodiments have been set forth only for the purposes of example and that it should not be taken as limiting this disclosure. It will be apparent to those skilled in the art that alterations, other embodiments, improvements, details and uses can be made consistent with the letter and spirit of this disclosure herein and within the scope of this invention, which is limited only by the claims, construed in accordance with the patent law, including the doctrine of equivalents. In the claims that follow, reference characters used to designate claim steps are provided for convenience of description only, and are not intended to imply any particular order for performing the steps.

In this document and in its claims, the verb “to comprise” and its conjugations is used in its non-limiting sense to mean that items following the word are included, but items not specifically mentioned are not excluded. Throughout this disclosure, the verb “to comprise” may be replaced by “to consist” or “to consist essentially of” and vice versa. In addition the verb “to consist” may be replaced by “to consist essentially of” meaning that a composition as defined herein may comprise additional component(s) than the ones specifically identified, the additional component(s) not altering the unique characteristic of this disclosure. In addition, reference to an element by the indefinite article “a” or “an” does not exclude the possibility that more than one of the elements is present, unless the context clearly requires that there be one and only one of the elements. The indefinite article “a” or “an” thus usually means “at least one.” Throughout this disclosure, unless explicitly stated otherwise, the articles “a” and “an” are preferably replaced by “at least two,” more preferably by “at least three,” even more preferably by “at least four,” even more preferably by “at least five,” even more preferably by “at least six,” most preferably by “at least seven.”

Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference. The full content of the priority application EP20190209 is also incorporated by reference to the same extent as if the priority application was specifically and individually indicated to be incorporated by reference.

The terms “recombinant” or “transgenic” or “metabolically engineered” or “genetically modified,” as used herein with reference to a cell or host cell are used interchangeably and indicates that the cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene, or are described to be transformed by the introduction of at least one heterologous or exogenous gene. Metabolically engineered or recombinant or transgenic cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one that has been produced by a recombinant cell. A “heterologous sequence” or a “heterologous nucleic acid,” as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host microorganism cell, wherein techniques may be applied that will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” cell or microorganism as used within the context of this disclosure refers to a cell or microorganism that is genetically modified.

The term “endogenous,” within the context of this disclosure refers to any polynucleotide, polypeptide or protein sequence that is a natural part of a cell and is occurring at its natural location in the cell chromosome and of which the control of expression has not been altered compared to the natural control mechanism acting on its expression. The term “exogenous” refers to any polynucleotide, polypeptide or protein sequence that originates from outside the cell under study and not a natural part of the cell or that is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “wild type” refers to the commonly known genetic or phenotypical situation as it occurs in nature.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, glucose, galactose, N-acetylglucosamine, glucosamine, mannose, xylose, N-acetylmannosamine, N-acetylneuraminic acid, N-glycolylneuraminic acid, a sialic acid, N-acetylgalactosamine, galactosamine, fucose, rhamnose, glucuronic acid, gluconic acid, fructose and polyols.

With the term polyol is meant an alcohol containing multiple hydroxyl groups. For example, glycerol, sorbitol, or mannitol.

The term “disaccharide” as used herein refers to a saccharide composed of two monosaccharide units. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal and fucopyranosyl-(1-4)-N-glycolylneuraminic acid (Fuc-(1-4)-Neu5Gc).

“Oligosaccharides” are glycan structures that are composed of three or more monosaccharide subunits that are linked to each other via glycosidic bonds in a linear or in a branched structure. “Oligosaccharide” as the term is used herein refers to a saccharide polymer containing a small number, typically three to twenty, of simple sugars, i.e., monosaccharides. Preferably, the oligosaccharide as described herein contains monosaccharides selected from the list as used herein above. Examples of oligosaccharides include but are not limited to Lewis-type antigen oligosaccharides, sialylated oligosaccharides, fucosylated oligosaccharides, chitosan, chitosan oligosaccharide, sulphated chitosan, acetylated chitosan, heparosan, chondroitin sulphate, glycosaminoglycan oligosaccharide, heparin, heparan sulphate, chondroitin sulphate, dermatan sulphate, hyaluronan or hyaluronic acid, keratan sulphate mammalian milk oligosaccharides and human milk oligosaccharides.

As used herein, “mammalian milk oligosaccharide” (MMO) refers to oligosaccharides such as but not limited to lacto-N-triose II, 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-neotetraose d, sialyllacto-N-neotetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-neotetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides. Mammalian milk oligosaccharides (MMOs) comprise oligosaccharides present in milk found in any phase during lactation including colostrum milk from humans (i.e., human milk oligosaccharides or HMOs) and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus). Human milk oligosaccharides (HMOs) are also known as human identical milk oligosaccharides that are chemically identical to the human milk oligosaccharides found in human breast milk but which are biotechnologically-produced (e.g., using cell free systems or cells and organisms comprising a bacterium, a fungus, a yeast, a plant, animal, or protozoan cell, preferably genetically engineered cells and organisms). Human identical milk oligosaccharides are marketed under the name HiMO.

As used herein, “lactose-based mammalian milk oligosaccharide (MMO)” refers to a MMO as defined herein that contains a lactose at its reducing end.

As used herein the term “Lewis-type antigens” comprise the following oligosaccharides: H1 antigen, which is Fucα1-2Galβ1-3GlcNAc, or in short 2′FLNB; Lewisa, which is the trisaccharide Galβ1-3[Fucα1-4]GlcNAc, or in short 4-FLNB; Lewisb, which is the tetrasaccharide Fucα1-2Galβ1-3[Fucα1-4]GlcNAc, or in short DiF-LNB; sialyl Lewisa, which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewisx, which is the trisaccharide Galβ1-4[Fucα1-3]GlcNAc, or otherwise known as 3-Fucosyl-N-acetyl-lactosamine, in short 3-FLacNAc, Lewisy, which is the tetrasaccharide Fucα1-2Galβ1-4[Fucα1-3]GlcNAc and sialyl Lewisx, which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, a ‘sialylated oligosaccharide’ is to be understood as a charged sialic acid containing oligosaccharide, i.e., an oligosaccharide having a sialic acid residue. It has an acidic nature. A sialylated oligosaccharide contains at least one sialic acid monosaccharide subunit, like e.g., but not limited to Neu5Ac, and Neu5Gc. The sialylated oligosaccharide is a saccharide structure comprising at least three monosaccharide subunits linked to each other via glycosidic bonds, wherein at least one of the monosaccharide subunit is a sialic acid. The sialylated oligosaccharide can contain more than one sialic acid residue, e.g., two, three or more. The sialic acid can be linked to other monosaccharide subunits comprising galactose, GlcNAc, sialic acid, via alpha-glycosidic bonds comprising alpha-2,3, alpha-2,6 linkages. Some examples are 3-SL (3′-sialyllactose or 3′-SL or Neu5Ac-a2,3-Gal-b1,4-Glc), 3′-sialyllactosamine, 6-SL (6′-sialyllactose or 6′-SL or Neu5Ac-a2,6-Gal-b1,4-Glc), 6′-sialyllactosamine, oligosaccharides comprising 6′-sialyllactose, 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, sialyllacto-N-neotetraose c, sialyllacto-N-neotetraose d, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1aNeu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Gal-1,4Glc, GQ1b Neu5Acα-2,8Neu5Acα-2,3Galβ-1,3GalNAcβ-1,4(Neu5Acα-2,8Neu5Acα2,3)Gal-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAcβ-1,4(Neu5Acα-2,8Neu5Acα-2,8Neu5Acα2,3)Gal-1,4Glc, GP1c Neu5Acα-2,8Neu5Acα-2,3Galβ-1,3GalNAcβ-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fucα-1,2Galβ-1,3GalNAcβ-1,4(Neu5Acα-2,3)Gal β-1,4Glc; all of which may be extended to the production of the corresponding gangliosides by reacting the above oligosaccharide moieties with ceramide or synthetizing the above oligosaccharides on a ceramide.

The terms “alpha-2,3-sialyltransferase,” “alpha 2,3 sialyltransferase,” “3-sialyltransferase, “α-2,3-sialyltransferase,” “α 2,3 sialyltransferase,” “3 sialyltransferase, “3-ST” or “3ST” as used in this disclosure, are used interchangeably and refer to a glycosyltransferase that catalyzes the transfer of sialic acid from the donor CMP-Neu5Ac, to the acceptor molecule in an alpha-2,3-linkage. The terms “3′ sialyllactose,” “3′-sialyllactose,” “alpha-2,3-sialyllactose,” “alpha 2,3 sialyllactose,” “α-2,3-sialyllactose,” “a 2,3 sialyllactose,” 3SL” or “3′SL” as used in this disclosure, are used interchangeably and refer to the product obtained by the catalysis of the alpha-2,3-fucosyltransferase transferring the sialic acid group from CMP-Neu5Ac to lactose in an alpha-2,3-linkage. The terms “alpha-2,6-sialyltransferase,” “alpha 2,6 sialyltransferase,” “6-sialyltransferase, “α-2,6-sialyltransferase,” “a 2,6 sialyltransferase,” “6 sialyltransferase, “6-ST” or “6ST” as used in this disclosure, are used interchangeably and refer to a glycosyltransferase that catalyzes the transfer of sialic acid from the donor CMP-Neu5Ac, to the acceptor molecule in an alpha-2,6-linkage. The terms “6′ sialyllactose,” “6′-sialyllactose,” “alpha-2,6-sialyllactose,” “alpha 2,6 sialyllactose,” “α-2,6-sialyllactose,” “a 2,6 sialyllactose,” 6SL” or “6′SL” as used in this disclosure, are used interchangeably and refer to the product obtained by the catalysis of the alpha-2,6-fucosyltransferase transferring the sialic acid group from CMP-Neu5Ac to lactose in an alpha-2,6-linkage. The terms “alpha-2,8-sialyltransferase,” “alpha 2,8 sialyltransferase,” “8-sialyltransferase, “α-2,8-sialyltransferase,” “a 2,8 sialyltransferase,” “8 sialyltransferase, “8-ST” or “8ST” as used in this disclosure, are used interchangeably and refer to a glycosyltransferase that catalyzes the transfer of sialic acid from the donor CMP-Neu5Ac, to the acceptor in an alpha-2,8-linkage.

A ‘fucosylated oligosaccharide’ as used herein and as generally understood in the state of the art is an oligosaccharide that is carrying a fucose-residue. Such fucosylated oligosaccharide is a saccharide structure comprising at least three monosaccharide subunits linked to each other via glycosidic bonds, wherein at least one of the monosaccharide subunit is a fucose. A fucosylated oligosaccharide can contain more than one fucose residue, e.g., two, three or more. A fucosylated oligosaccharide can be a neutral oligosaccharide or a charged oligosaccharide e.g., also comprising sialic acid structures. Fucose can be linked to other monosaccharide subunits comprising glucose, galactose, GlcNAc via alpha-glycosidic bonds comprising alpha-1,2 alpha-1,3, alpha-1,4, alpha-1,6 linkages.

Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNFP I), Lacto-N-fucopentaose II (LNFP II), Lacto-N-fucopentaose III (LNFP III), lacto-N-fucopentaose V (LNFP V), lacto-N-fucopentaose VI (LNFP VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose.

The terms “alpha-1,2-fucosyltransferase,” “alpha 1,2 fucosyltransferase,” “2-fucosyltransferase, “α-1,2-fucosyltransferase,” “a 1,2 fucosyltransferase,” “2 fucosyltransferase, “2-FT” or “2FT” as used in this disclosure, are used interchangeably and refer to a glycosyltransferase that catalyzes the transfer of fucose from the donor GDP-L-fucose, to the acceptor molecule in an alpha-1,2-linkage. The terms “2′ fucosyllactose,” “2′-fucosyllactose,” “alpha-1,2-fucosyllactose,” “alpha 1,2 fucosyllactose,” “α-1,2-fucosyllactose,” “α 1,2 fucosyllactose,” “Galβ-4(Fucα1-2)Glc,” 2FL” or “2′FL” as used in this disclosure, are used interchangeably and refer to the product obtained by the catalysis of the alpha-1,2-fucosyltransferase transferring the fucose residue from GDP-L-fucose to lactose in an alpha-1,2-linkage. The terms “difucosyllactose,” “di-fucosyllactose,” “lactodifucotetraose,” “2′,3-difucosyllactose,” “2′,3 difucosyllactose,” “α-2′,3-fucosyllactose,” “a 2′,3 fucosyllactose, “Fucα1-2Galβ 1-4(Fucα1-3)Glc,” “DFLac,” 2′,3 diFL,” “DFL,” “DiFL” or “diFL” as used in this disclosure, are used interchangeably.

The terms “alpha-1,3-fucosyltransferase,” “alpha 1,3 fucosyltransferase,” “3-fucosyltransferase, “α-1,3-fucosyltransferase,” “a 1,3 fucosyltransferase,” “3 fucosyltransferase, “3-FT” or “3FT” as used in this disclosure, are used interchangeably and refer to a glycosyltransferase that catalyzes the transfer of fucose from the donor GDP-L-fucose, to the acceptor molecule in an alpha-1,3-linkage. The terms “3-fucosyllactose,” “alpha-1,3-fucosyllactose,” “alpha 1,3 fucosyllactose,” “α-1,3-fucosyllactose,” “a 1,3 fucosyllactose,” “Galβ-4(Fucα1-3)Glc,” 3FL” or “3-FL” as used in this disclosure, are used interchangeably and refer to the product obtained by the catalysis of the alpha-1,3-fucosyltransferase transferring the fucose residue from GDP-L-fucose to lactose in an alpha-1,3-linkage.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′, 3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

The terms “LNB” and “Lacto-N-biose” are used interchangeably and refer to the disaccharide Gal-b1,3-GlcNAc.

The terms “LacNAc” and “N-acetyllactosamine” are used interchangeably and refer to the disaccharide Gal-b1,4-GlcNAc.

The terms “LNT II,” “LNT-II,” “LN3,” “lacto-N-triose II,” “lacto-N-triose II,” “lacto-N-triose,” “lacto-N-triose” or “GlcNAcβ1-3Galβ1-4Glc” as used in this disclosure, are used interchangeably.

The terms “LNT,” “lacto-N-tetraose,” “lacto-N-tetraose” or “Galβ1-3GlcNAcβ1-3Galβ1-4GlC” as used in this disclosure, are used interchangeably.

The terms “LNnT,” “lacto-N-neotetraose,” “lacto-N-neotetraose,” “neo-LNT” or “Galβ1-4GlcNAcβ1-3Galβ1-4GlC” as used in this disclosure, are used interchangeably.

The terms “LSTa,” “LS-Tetrasaccharide a,” “Sialyl-lacto-N-tetraose a,” “sialyllacto-N-tetraose a” or “Neu5Ac-a2,3-Gal-b1,3-GlcNAc-b1,3-Gal-b1,4-Glc” as used in this disclosure, are used interchangeably.

The terms “LSTb,” “LS-Tetrasaccharide b,” “Sialyl-lacto-N-tetraose b,” “sialyllacto-N-tetraose b” or “Gal-b1,3-(Neu5Ac-a2,6)-GlcNAc-b1,3-Gal-b1,4-Glc” as used in this disclosure, are used interchangeably.

The terms “LSTc,” “LS-Tetrasaccharide c,” “Sialyl-lacto-N-tetraose c,” “sialyllacto-N-tetraose c,” “sialyllacto-N-neotetraose c” or “Neu5Ac-a2,6-Gal-b1,4-GlcNAc-b1,3-Gal-b1,4-Glc” as used in this disclosure, are used interchangeably.

The terms “LSTd,” “LS-Tetrasaccharide d,” “Sialyl-lacto-N-tetraose d,” “sialyllacto-N-tetraose d,” “sialyllacto-N-neotetraose d” or “Neu5Ac-a2,3-Gal-b1,4-GlcNAc-b1,3-Gal-b1,4-Glc” as used in this disclosure, are used interchangeably.

The terms “DSLNnT” and “Disialyllacto-N-neotetraose” are used interchangeably and refer to Neu5Ac-a2,6-[Neu5Ac-a2,6-Gal-b1,4-GlcNAc-b1,3]-Gal-b1,4-Glc.

The terms “DSLNT” and “Disialyllacto-N-tetraose” are used interchangeably and refer to Neu5Ac-a2,6-[Neu5Ac-a2,3-Gal-b1,3-GlcNAc-b1,3]-Gal-b1,4-Glc. The terms “LNFP-I,” “lacto-N-fucopentaose I,” “LNFP I,” “LNF I OH type I determinant,” “LNF I,” “LNF1,” “LNF 1” and “Blood group H antigen pentaose type 1” are used interchangeably and refer to Fuc-a1,2-Gal-b1,3-GlcNAc-b1,3-Gal-b1,4-Glc.

The terms “GalNAc-LNFP-I” and “blood group A antigen hexaose type I” are used interchangeably and refer to GalNAc-a1,3-(Fuc-a1,2)-Gal-b1,3-GlcNAc-b1,3-Gal-b1,4-Glc.

The terms “LNFP-II” and “lacto-N-fucopentaose II” are used interchangeably and refer to Gal-b1,3-(Fuc-a1,4)-GlcNAc-b1,3-Gal-b1,4-Glc.

The terms “LNFP-III” and “lacto-N-fucopentaose III” are used interchangeably and refer to Gal-b1,4-(Fuc-a1,3)-GlcNAc-b1,3-Gal-b1,4-Glc.

The terms “LNFP-V” and “lacto-N-fucopentaose V” are used interchangeably and refer to Gal-b1,3-GlcNAc-b1,3-Gal-b1,4-(Fuc-a1,3)-Glc.

The terms “LNFP-VI,” “LNnFP V” and “lacto-N-neofucopentaose V” are used interchangeably and refer to Gal-b1,4-GlcNAc-b1,3-Gal-b1,4-(Fuc-a1,3)-Glc.

The terms “LNnFP I” and “Lacto-N-neofucopentaose I” are used interchangeably and refer to Fuc-a1,2-Gal-b1,4-GlcNAc-b1,3-Gal-b1,4-Glc.

The terms “LNDFH I,” “Lacto-N-difucohexaose I,” “LNDFH-I,” “LDFH I,” “Le^{b}-lactose” and “Lewis-b hexasaccharide” are used interchangeably and refer to Fuc-a1,2-Gal-b1,3-[Fuc-a1,4]-GlcNAc-b1,3-Gal-b1,4-Glc.

The terms “LNDFH II,” “Lacto-N-difucohexaose II,” “Lewis a-Lewis x” and “LDFH II” are used interchangeably and refer to Fuc-a1,4-(Gal-b1,3)-GlcNAc-b1,3-Gal-b1,4-(Fuc-a1,3)-Glc.

The terms “LNnDFH,” “Lacto-N-neoDiFucohexaose” and “Lewis x hexaose” are used interchangeably and refer to Gal-b1,4-(Fuc-a1,3)-GlcNAc-b1,3-Gal-b1,4-(Fuc-a1,3)-Glc.

The terms “alpha-tetrasaccharide” and “A-tetrasaccharide” are used interchangeably and refer to GalNAc-a1,3-(Fuc-a1,2)-Gal-b1,4-Glc.

“Charged oligosaccharides” are oligosaccharide structures that contain one or more negatively charged monosaccharide subunits including N-acetylneuraminic acid (Neu5Ac), commonly known as sialic acid, N-glycolylneuraminic acid (Neu5Gc), glucuronate and galacturonate. Charged oligosaccharides are also referred to as acidic oligosaccharides. Sialic acid belongs to the family of derivatives of neuraminic acid (5-amino-3,5-dideoxy-D-glycero-D-galacto-non-2-ulosonic acid). Neu5Gc is a derivative of sialic acid, which is formed by hydroxylation of the N-acetyl group at C5 of Neu5Ac. In contrast, neutral oligosaccharides are non-sialylated oligosaccharides, and thus do not contain an acidic monosaccharide subunit. Neutral oligosaccharides comprise non-charged fucosylated oligosaccharides that contain one or more fucose subunits in their glycan structure as well as non-charged non-fucosylated oligosaccharides that lack any fucose subunit. Other examples of charged oligosaccharides are sulphated chitosans and deacetylated chitosans.

As used herein, an antigen of the human ABO blood group system is an oligosaccharide. Such antigens of the human ABO blood group system are not restricted to human structures. The structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal—that are present on disaccharide core structures comprising Gal-beta1,3-GlcNAc, Gal-beta1,4-GlcNAc, Gal-beta1,3-GalNAc and Gal-beta1,4-Glc.

A ‘fucosylation pathway’ as used herein is a biochemical pathway comprising the enzymes and their respective genes, mannose-6-phosphate isomerase, phosphomannomutase, mannose-1-phosphate guanylyltransferase, GDP-mannose 4,6-dehydratase, GDP-L-fucose synthase and/or the salvage pathway L-fucokinase/GDP-fucose pyrophosphorylase, combined with a fucosyltransferase leading to α 1,2; α 1,3 α 1,4 and/or α 1,6 fucosylated oligosaccharides.

A ‘sialylation pathway’ is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, Glucosamine 6-phosphate N-acetyltransferase, N-AcetylGlucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, and/or CMP-sialic acid synthase, combined with a sialyltransferase leading to α 2,3; α 2,6 and/or α 2,8 sialylated oligosaccharides.

A ‘galactosylation pathway’ as used herein is a biochemical pathway comprising the enzymes and their respective genes, galactose-1-epimerase, galactokinase, glucokinase, galactose-1-phosphate uridylyltransferase, UDP-glucose 4-epimerase, glucose-1-phosphate uridylyltransferase, and/or glucophosphomutase, combined with a galactosyltransferase leading to an alpha or beta bound galactose on the 2, 3, 4, and/or 6 hydroxyl group of an oligosaccharide.

An ‘N-acetylglucosamine carbohydrate pathway’ as used herein is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridylyltransferase, glucosamine-1-phosphate acetyltransferase, and/or glucosamine-1-phosphate acetyltransferase, combined with a glycosyltransferase leading to an alpha or beta bound N-acetylglucosamine on the 3, 4, and/or 6 hydroxylgroup of an oligosaccharide.

An ‘N-acetylgalactosaminylation pathway’ as used herein is a biochemical pathway comprising at least one of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and/or UDP-N-acetylgalactosamine pyrophosphorylase combined with a glycosyltransferase leading to a GalNAc-modified compound comprising a mono-, di- or oligosaccharide having an alpha or beta bound N-acetylgalactosamine on the mono-, di- or oligosaccharide.

A ‘mannosylation pathway’ as used herein is a biochemical pathway comprising at least one of the enzymes and their respective genes chosen from the list comprising mannose-6-phosphate isomerase, phosphomannomutase and/or mannose-1-phosphate guanylyltransferase combined with a glycosyltransferase leading to a mannosylated compound comprising a mono-, di- or oligosaccharide having an alpha or beta bound mannose on the mono-, di- or oligosaccharide.

An ‘N-acetylmannosaminylation pathway’ as used herein is a biochemical pathway comprising at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to a ManNAc-modified compound comprising a mono-, di- or oligosaccharide having an alpha or beta bound N-acetylmannosamine on the mono-, di- or oligosaccharide.

The term “consisting essentially of” as used herein and as used in the art, refers to compositions consisting of the compound(s) specified after the term, and—optionally—inevitable by-products. The inevitable by-products include—for example—compounds that were generated during cultivation or microbial fermentation for the production of the mixture of oligosaccharides as well as compounds that were introduced into a process stream from which the oligosaccharide mixture is recovered, but which could not have been removed therefrom.

The term “consisting essentially of” with respect to spray-dried powders includes spray-dried powders containing with respect to the dry matter of the spray-dried powder at least 80%-wt., at least 85%-wt., at least 90%-wt., at least 93%-wt., at least 95%-wt. or at least 98%-wt. the oligosaccharide mixture. The term “consisting essentially of” is used likewise with respect to spray-dried powders, process streams and solutions containing the oligosaccharide mixture.

Further herein, the terms “contaminants” and “impurities” preferably mean particulates, cells, cell components, metabolites, cell debris, proteins, peptides, amino acids, nucleic acids, glycolipids and endotoxins that can be present in an aqueous medium from a cultivation or fermentation process.

The term “clarifying” as used herein refers to the act of treating an aqueous medium or cultivation or fermentation broth to remove suspended particulates and contaminants from the cultivation or fermentation process, particularly cells, cell components, insoluble metabolites and debris, that could interfere with the eventual purification of the oligosaccharide mixture. Such treatment can be carried out in a conventional manner by centrifugation, flocculation, flocculation with optional ultrasonic treatment, gravity filtration, microfiltration, foam separation or vacuum filtration (e.g., through a ceramic filter that can include a CELITE™ filter aid).

The terms “protein-free oligosaccharide mixture” as used herein means an oligosaccharide mixture from an aqueous medium or broth from a cultivation or fermentation process, which medium has been treated to remove substantially all the proteins, as well as any related impurities, such as amino acids, peptides, endotoxins, glycolipids, RNA and DNA, from the process that could interfere with the eventual purification of the oligosaccharide mixture from the process. Such removal of proteins, preferably substantially all proteins, can be accomplished in a conventional manner by ion exchange chromatography, affinity chromatography, ultrafiltration, and size exclusion chromatography. Preferably, a protein-free oligosaccharide mixture is a clarified oligosaccharide mixture.

The terms “purifying the oligosaccharide mixture from the cultivation or fermentation broth,” according to this disclosure, mean harvesting, collecting or retrieving the oligosaccharide mix from the cells and/or the medium of its growth. The term “cultivation” refers to the culture medium wherein the cell is cultivated or cultured or fermented, the cell itself, and the oligosaccharides that are produced by the cell in whole broth, i.e., inside (intracellularly) as well as outside (extracellularly) of the cell.

In case the oligosaccharide mix is still present or partly present in the cells producing the oligosaccharide mix, conventional manners to free or to extract the oligosaccharide mix out of the cells can be used, such as cell destruction using high pH, heat shock, sonication, French press, homogenization, enzymatic hydrolysis, chemical hydrolysis, solvent hydrolysis, detergent, hydrolysis. The culture medium and/or cell extract together and/or separately can then be further used for purifying the oligosaccharide mixture from the cultivation or fermentation broth.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of this disclosure are at least about 50.0%, 55.0%, 60.0%, 65.0%, 70.0%, 75.0%, 80.0% or 85.0% pure, usually at least about 90.0%, 91.0%, 92.0%, 93.0%, 94.0%, 95.0%, 96.0%, 97.0%, 98.0%, or 99.0% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, purity can be determined using methods such as but not limited to thin layer chromatography, gas chromatography, NMR, HPLC, capillary electrophoresis or mass spectroscopy.

A “solution comprising the purified mixture of oligosaccharides” comprises a mixture of oligosaccharides dissolved in an aqueous medium. An aqueous medium is a solvent comprising water. In some embodiments, the aqueous medium is pure water. In other embodiments, the medium comprises water with a trace amount of one or more organic solvents. In some such embodiments, the medium comprises less than 1%-wt. organic solvent. In some embodiments, the medium comprises less than 0.1%-wt. organic solvent. In some embodiments, the medium comprises less than 0.01%-wt. organic solvent. In some embodiments, the medium comprises less than 0.001%-wt. organic solvent. In some embodiments, the medium comprises less than 0.0001%-wt. organic solvent.

In some embodiments, the oligosaccharide mixture solution comprises a trace amount of one or more organic solvents. In some such embodiments, the oligosaccharide mixture solution comprises less than 1%-wt. organic solvent. In some embodiments, the oligosaccharide mixture solution comprises less than 0.1%-wt. organic solvent. In some embodiments, the oligosaccharide mixture solution comprises less than 0.01%-wt. (percent by weight) organic solvent. In some embodiments, the oligosaccharide mixture solution comprises less than 0.001%-wt. organic solvent. In some embodiments, the oligosaccharide mixture solution comprises less than 0.0001%-wt. organic solvent.

As used herein a “Brix value” indicates the sugar content of an aqueous solution. A Brix value can be expressed as a percentage (percent Brix) or as “degrees Brix” (degrees Brix). Strictly, a Brix value is the percentage by weight of sucrose in a pure water solution, and so does not apply to solutions comprising other solutes and/or solvents. However, a Brix value is simple to measure, and, therefore, is commonly used in the art as an approximation of the total saccharide content of sugar solutions other than pure sucrose solutions. As used herein, the “Brix value” indicates the combined sugar content of the aqueous solution.

Techniques for measuring a Brix value are well known in the art. Dissolution of sugar in an aqueous solution changes the refractive index of the solution. Accordingly, an appropriately calibrated refractometer can be used to measure a Brix value of a solution.

Alternatively, the density of a solution may be measured and converted to a Brix value. A digital density meter can perform this measurement and conversion automatically, or a hydrometer or pycnometer may be used.

As used herein, the term “bulk density” is the weight of the particles of a particulate solid (such as a powder) in a given volume, and is expressed in grams per liter (g/L). The total volume that the particles of a particulate solid occupy depends on the size of the particles themselves and the volume of the spaces between the particles. Entrapped air between and inside the particles also can affect the bulk density. Thus, a particulate solid comprising large, porous particles with large inter-particulate spaces will have a lower bulk density than a particulate solid comprising small, non-porous particles that compact closely together. Bulk density can be expressed in two forms: “loose bulk density” and “tapped bulk density.” Loose bulk density (also known in the art as “freely settled” or “poured” bulk density) is the weight of a particulate solid divided by its volume where the particulate solid has been allowed to settle into that volume of its own accord (e.g., a powder poured into a container).

Closer compaction of a particulate solid within a container may be achieved by tapping the container and allowing the particles to settle more closely together, thereby reducing volume while weight remains the same. Tapping therefore increases bulk density. Tapped bulk density (also known in the art as “tamped” bulk density) is the weight of a particulate solid divided by its volume where the particulate solid has been tapped and allowed to settle into the volume a precise number of times. The number of times the particulate solid has been tapped is typically when stating the tapped bulk density. For example, “100× tapped bulk density” refers to the bulk density of the particulate solid after it has been tapped 100 times.

Techniques for measuring bulk density are well known in the art. Loose bulk density may be measured using a measuring cylinder and weighing scales: the particulate solid is poured into the measuring cylinder and the weight and volume of the particulate solid; weight divided by volume gives the loose bulk density. Tapped bulk density can be measured using the same technique, with the addition of tapping the measuring cylinder a set number of times before measuring weight and volume. Automation of tapping ensures the number, timing and pressure of individual taps is accurate and consistent. Automatic tapping devices are readily available, an example being the Jolting Stampfvolumeter (STAV 203) from J. Englesmann AG.

The term “different oligosaccharides” as used herein refers to oligosaccharides that are structurally distinct.

The terms “dry solid” and “dry matter” as used herein are used interchangeably and are further described in Example 1.

Throughout this disclosure, unless explicitly stated otherwise, a “genetically modified micro-organism” or “metabolically engineered micro-organism” or “genetically modified cell” or “metabolically engineered cell” preferably means respectively, a microorganism or a cell that is genetically modified or metabolically engineered, respectively, for the production of the mixture comprising different oligosaccharides according to this disclosure. In the context of this disclosure, the different oligosaccharides of the mixture as disclosed herein preferably do not occur in the wild type progenitor of the metabolically engineered micro-organism or cell, respectively.

Throughout this disclosure, unless explicitly stated otherwise, the features “synthesize,” “synthesized” and “synthesis” are interchangeably used with the features “produce,” “produced” and “production,” respectively.

### DETAILED DESCRIPTION

According to a first aspect, a method to produce a purified mixture of different oligosaccharides produced by at least one cell, preferably a single cell, preferably a cell of a micro-organism, is provided that synthesizes the mixture of different oligosaccharides. The method comprises culturing at least one cell, preferably of a micro-organism, that synthesizes a mixture of different oligosaccharides in a suitable cultivation or fermentation medium to form a cultivation or fermentation broth, and then purifying the oligosaccharide mixture from the cultivation or fermentation broth. The purification comprises a combination of clarification of the cultivation or fermentation broth and removing salts and/or medium components from the clarified cultivation or fermentation broth and/or concentrating the oligosaccharide mixture in the clarified cultivation or fermentation broth thereby providing a solution comprising the purified mixture of oligosaccharides. In an embodiment, the clarification is combined with the removal of salts and/or medium components. In an embodiment, the clarification is combined with the step of concentrating the oligosaccharide mixture in the clarified cultivation or fermentation broth. In an embodiment, the clarification is combined with the removal of salts and/or medium components and further combined with the step of concentrating the oligosaccharide mixture resulting from the step of removal of salts and/or medium components. In an embodiment, the clarification is combined with the step of concentrating the oligosaccharide mixture and further combined with the removal of salts and/or medium components of the oligosaccharide mixture resulting from the step of concentrating.

The method of this disclosure allows efficient purification of large quantities of a mix of oligosaccharides at high purity.

Contrary to the purification currently used in cell cultivation or fermentation for the production of oligosaccharides that provide for the separation of single oligosaccharides, the present method allows the provision of a purified mixture of different oligosaccharides. The so purified solution of oligosaccharide mixture may be obtained in solid form by drying, preferably spray drying, lyophilization or concentrated to a syrup of at least 40% dry matter. The provided oligosaccharides are free of proteins and recombinant material originating from the used recombinant cell cultivation or microbial strains and thus very well-suited for use in food, medical food and feed (e.g., pet food) applications.

In an embodiment, the purification involves clarifying the oligosaccharide mix containing cultivation or fermentation broth to remove suspended particulates and contaminants, particularly cells, cell components, insoluble metabolites and debris produced by culturing the genetically modified cell. In this step, the cultivation or fermentation broth containing the produced oligosaccharide mixture can be clarified in a conventional manner. Preferably, the oligosaccharide mix containing mixture is clarified by centrifugation, flocculation, decantation, ultrafiltration and/or filtration. A second step of purifying the oligosaccharide mixture from the cultivation or fermentation broth preferably involves removing salts and/or medium components, comprising proteins, as well as peptides, amino acids, RNA and DNA and any endotoxins and glycolipids that could influence purity, from the cultivation or fermentation broth containing the oligosaccharide mixture, after it has been clarified. In this step, proteins, salts, by-products, color and other related impurities are removed from the oligosaccharide mix containing mixture by ultrafiltration, nanofiltration, reverse osmosis, microfiltration, activated charcoal or carbon treatment, tangential flow high-performance filtration, tangential flow ultrafiltration, affinity chromatography, ion exchange (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while the oligosaccharide mix remains in the oligosaccharide mix containing mixture. A third step of purifying the oligosaccharide mixture from the cultivation or fermentation broth preferably involves concentrating the cultivation or fermentation broth containing the oligosaccharide mixture. In an embodiment, the third step precedes the second step. In an embodiment, the step of concentrating precedes the second step and is once more applied after the second step as described above.

In an embodiment, the oligosaccharide mixture comprises at least 2 different oligosaccharides, preferably at least 3 different oligosaccharides, more preferably at least 4 different oligosaccharides, even more preferably at least 5 different oligosaccharides, most preferably at least 6 different oligosaccharides.

Such an oligosaccharide mixture comprising different oligosaccharides can comprise, for example, 5 structurally different oligosaccharides such as e.g., 2′-FL, 3-FL, LNT, 3′-SL and 6′-SL; another example comprises 7 structurally different oligosaccharides such as e.g., 2′-FL, 3-FL, LNT, LNnT, LNFPI, 3′-SL and 6′-SL.

In another preferred embodiment, the oligosaccharide mixture comprises at least 2 different oligosaccharides that differ in degree of polymerization (DP), preferably the oligosaccharide mixture comprises at least 3 different oligosaccharides that differ in degree of polymerization, more preferably the oligosaccharide mixture comprises at least 4 different oligosaccharides that differ in degree of polymerization. The degree of polymerization of an oligosaccharide refers to the number of monosaccharide units present in the oligosaccharide structure. As used herein, the degree of polymerization of an oligosaccharide is three (DP3) or more, the latter comprising any one of 4 (DP4), 5 (DP5), 6 (DP6) or longer. The oligosaccharide mixture as described herein preferably comprises at least three different oligosaccharides wherein all oligosaccharides present in the mixture have a different degree of polymerization from each other. For example, the oligosaccharide mixture can comprise three oligosaccharides, wherein the first oligosaccharide is a trisaccharide with a degree of polymerization of 3 (DP3), the second oligosaccharide is a tetrasaccharide with a degree of polymerization of 4 (DP4) and the third oligosaccharide is a pentasaccharide with a degree of polymerization of 5 (DP5).

In an embodiment, an oligosaccharide mixture comprising two different oligosaccharides that differ in degree of polymerization is a mixture of 2′FL and LNT; mix of 2′FL and DiFL or a mixture of 2′FL and LNFPI. In an embodiment an oligosaccharide mixture comprising three different oligosaccharides that differ in DP is a mixture of 2′FL, DiFL and LNFPI.

In an embodiment, the at least one cell produces a mixture comprising four different oligosaccharides or more than four different oligosaccharides. Such mixture can comprise at least four different oligosaccharides wherein three of the oligosaccharides have a different degree of polymerization. Alternatively, all of the oligosaccharides in the mixture can have a different degree of polymerization as described herein.

Alternatively or preferably, the oligosaccharide mixture comprises at least one neutral and at least one charged oligosaccharide.

The at least one cell used for the production of the mixture of oligosaccharides can be a fungal, yeast, bacterial, insect, animal, and plant and protozoan cell. Preferably, the cells used are cells of a micro-organism.

Preferably, the at least one cell that synthesizes a mixture of different oligosaccharides comprises at least one metabolically engineered cell that is metabolically engineered for the production of the mixture.

More preferably, only one type of cell is used that is producing the mixture.

More preferably, only one cell is used that is metabolically engineered for the production of the mixture.

According to an embodiment of this disclosure, the mixture of oligosaccharides in the cultivation or fermentation broth is obtained by culturing at least one genetically modified cell capable of producing the mixture of oligosaccharides, preferably from an internalized carbohydrate precursor.

In an embodiment, at least one of the cells has been genetically modified to produce at least one oligosaccharide, preferably the at least one cell has been genetically modified to produce at least two different oligosaccharides.

According to an embodiment of this disclosure, the mixture of oligosaccharides in the cultivation or fermentation broth is obtained by culturing at least one genetically modified cell capable of producing the mixture of oligosaccharides, preferably from an internalized carbohydrate precursor.

According to an embodiment of this disclosure, the mixture of oligosaccharides in the cultivation or fermentation broth is obtained by culturing at least one genetically modified cell or microorganism capable of producing the mixture of oligosaccharides, preferably from an internalized carbohydrate precursor.

In an embodiment, at least one of the cell has been genetically modified to produce at least one oligosaccharide, preferably the at least one cell has been genetically modified to produce at least two different oligosaccharides.

In an embodiment, at least one of the micro-organism has been genetically modified to produce at least one oligosaccharide, preferably the at least one micro-organism has been genetically modified to produce at least two different oligosaccharides.

In an embodiment, the cultivation or fermentation broth comprises the mixture of oligosaccharides, biomass, medium components and contaminants.

According to an embodiment of this disclosure, the at least one cell, preferably cell of a micro-organism, is cultured in a minimal salt medium with a carbon source on which the at least one micro-organism grows. Preferably, the minimal salt medium contains sulphate, phosphate, chloride, ammonium, calcium ion, magnesium ion, sodium ion, potassium ion, iron ion, copper ion, zinc ion, manganese ion, cobalt ion, and/or selenium ion.

The at least one cell, preferably a cell of a micro-organism, as used herein grows on a monosaccharide, disaccharide, oligosaccharide, polysaccharide, polyol, a complex medium or a mixture thereof as the main carbon source. With the term main is meant the most important carbon source for the bioproducts of interest, biomass formation, carbon dioxide and/or by-products formation (such as acids and/or alcohols, such as acetate, lactate, and/or ethanol), i.e., 20.0%, 25.0%, 30.0%, 35.0%, 40.0%, 45.0%, 50.0%, 55.0%, 60.0%, 65.0%, 70.0%, 75.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100% of all the required carbon is derived from the above-indicated carbon source. In an embodiment of this disclosure, the carbon source is the sole carbon source for the organism, i.e., 100% of all the required carbon is derived from the above-indicated carbon source. Common main carbon sources comprise but are not limited to glucose, glycerol, fructose, maltose, lactose, arabinose, malto-oligosaccharides, maltotriose, sorbitol, xylose, rhamnose, sucrose, galactose, mannose, methanol, ethanol, trehalose, starch, cellulose, hemi-cellulose, corn-steep liquor, high-fructose syrup, acetate, citrate, lactate and pyruvate. With the term complex medium is meant a medium for which the exact constitution is not determined. Examples are molasses, corn steep liquor, peptone, tryptone or yeast extract.

Alternatively or preferably, the carbon source comprises one or more of glucose, fructose, mannose, sucrose, maltose, corn steep liquor, lactose, galactose, high fructose syrup, starch, cellulose, hemi-cellulose, malto-oligosaccharides, trehalose, glycerol, acetate, citrate, lactate and pyruvate.

According to a preferred embodiment of this disclosure, the combined purity of the mixture of oligosaccharides in the cultivation or fermentation broth is less than 80% on total dry solid, and/or the combined purity of the purified mixture of oligosaccharides in the solution is equal to or more than 80% on total dry solid.

As used herein “the combined purity of the mixture of oligosaccharides” refers to the oligosaccharide part in the total cultivation or fermentation broth or in the purified solution containing the oligosaccharide mixture, before or after the purification steps, respectively.

Preferably, the combined purity of the mixture of oligosaccharides in the cultivation or fermentation broth is <70%, <60%, <50%, <40%, <30%, <20%, <10% on total dry solid, before the purification and/or the combined purity of the purified mixture of oligosaccharides is >80%, preferably of >85% or more preferably >90%, even more preferably >95%, most preferably >97% on total dry solid after the purification.

In an embodiment, the solution comprising the purified mixture of oligosaccharides has a combined purity of at least 80%, at least 85%, at least 90%, at least 93%, at least 95% or at least 98% with respect to the weight of dry matter/solutes within the solution.

In an embodiment, the yield of the purification of the oligosaccharide solution is >60%, preferably >65%, more preferably >70%, most preferably >75%. The yield being calculated on the basis of the total mass of oligosaccharide or oligosaccharides in the final syrup or powder divided by the total mass of oligosaccharide or oligosaccharides in the broth after clarification in percentage.

According to an aspect of this disclosure, the step i) of clarifying the cultivation or fermentation broth comprises one or more of clarification, clearing, filtration, microfiltration, centrifugation, decantation and ultrafiltration, preferably the step i) further comprising use of a filter aid and/or flocculant. Alternatively or preferably, step i) comprises subjecting the cultivation or fermentation broth to two membrane filtration steps using different membranes. Further alternatively or preferably, step i) of clarifying the cultivation or fermentation broth further comprises use of a filtration aid, preferably an adsorbing agent, more preferably active carbon.

In an aspect of this disclosure, step ii) of removing salts and/or medium components from the clarified cultivation or fermentation broth comprises at least one or more of nanofiltration, dialysis, electrodialysis, use of activated charcoal or carbon, use of solvents, use of alcohols, and use of aqueous alcohol mixtures, use of charcoal, tangential flow high-performance filtration, tangential flow ultrafiltration, affinity chromatography, ion exchange, ion exchange chromatography, hydrophobic interaction chromatography, gel filtration, ligand exchange chromatography, column chromatography, cation exchange adsorbent resin, and use of ion exchange resin. Preferably, step ii) of removing salts and/or medium components from the clarified cultivation or fermentation broth by ion exchange is any one or more of cation exchange, anion exchange, mixed bed ion exchange, simulated moving bed chromatography.

In an embodiment, step ii) of removing salts and/or medium components from the clarified cultivation or fermentation broth comprises anion exchange wherein the anion exchange resin has a moisture content of 30-48% and preferably is a gel type anion exchanger. Such anion exchanger is preferably selected from the group comprising DOWEX™ 1-X8, XA4023, XA3112, DIAION™ SA20A, DIAION™ SA10A, preferably in OH— form. Such anion exchange treatment is very performant for oligosaccharide mixture solution purification wherein the oligosaccharide mixture comprises charged oligosaccharide, especially sialylated oligosaccharides such as sialyllactose. As such, such anion exchange resin can be used in a pure anion exchange step combined with a cation exchange step or used in a mixed bed ion exchange setting.

In an embodiment, step ii) comprises a step of cation exchange combined with a step of anion exchange wherein the anion exchange resin has a moisture content of 30-48% and preferably is a gel type anion exchanger, preferably as described herein. In an embodiment, the step of cation exchange precedes the step of anion exchange.

The anion exchange resin, characterized by the moisture content of 30-48 percent, is preferably a gel type anion exchanger that desalts the clarified cultivation or fermentation broth, though without thereby binding the charged, e.g., sialyl, group containing oligosaccharides and, in particular, the sialyllactose, which oligosaccharides are also present in salt form. In other words, this involves an anion exchange resin that has selectivity for negatively charged minerals, but not for sialyllactose. As described in the art, see e.g., WO 2009/113861, to this end, it is necessary that the moisture content, that is, the water content, is not greater than 48%, and preferably not greater than 45%. At moisture contents lower than 35%, and more so at moisture contents lower than 30%, the desalting capacity starts to become too low to yield an effective process. The moisture content in the anion exchanger is determined in the following manner: prior to measurement of the moisture content of the resin, adhering water is removed, for instance, by wrapping the resin in a cloth and then subjecting it to centrifugation (centrifuge: 30 cm diameter; 3,000 rpm); the resin is then weighed, for instance, in a weighing bottle; after which the resin is dried for 4 hours at a constant temperature of 105° C.; the resin is then cooled down in an exicator for 30 minutes; after which in turn the weight of the dry resin is determined; the moisture percentage (weight percent)=[(weight loss after drying (g))/(weight of the wet resin)]* 100 percent. Through this desalting, an important part of the negatively charged ions is removed without substantial amounts of sialyllactose (despite the negative charge) being thereby removed.

The anion exchange resin mentioned is preferably and usually in the free base form (hydroxide form) because this results in a greatest possible desalting capacity. Suitable anion exchange resins are strongly cross-linked polystyrene-divinylbenzene gels, such as DIAION™ SA20A or DIAION™ WA20A.

In an embodiment, step ii) comprises a treatment with a mixed bed ion exchange resin. In an embodiment, such mixed bed ion exchange resin is a mixed bed column of DIAION™ SA20A and AMBERLITE™ FPC 22H mixed in a ratio 1.1:1 to 1.9:1. In an embodiment, such mixed bed ion exchange resin comprises an anion exchange resin having a moisture content of 30-48% and preferably being microporous or a gel type anion exchanger. As explained above, such anion exchange type is very useful in the purification of solutions comprising charged oligosaccharide.

In another aspect, step iii) of concentrating comprises one or more of nanofiltration, diafiltration, reverse osmosis, evaporation, wiped film evaporation, and falling film evaporation.

In an embodiment, the mixture of oligosaccharides comprises at least one of a fucosylated oligosaccharide, sialylated oligosaccharide, Lewis type antigen, an N-acetylglucosamine containing neutral oligosaccharide, N-acetyllactosamine containing oligosaccharide, lacto-N-biose containing oligosaccharide, non-fucosylated neutral oligosaccharide, chitosan, chitosan oligosaccharide, heparosan, chondroitin sulphate, glycosaminoglycan oligosaccharide, heparin, heparan sulphate, chondroitin sulphate, dermatan sulphate, hyaluronan or hyaluronic acid and/or keratan sulphate. Alternatively or preferably, the mixture of oligosaccharides comprises at least one mammalian milk oligosaccharide, preferably at least one human milk oligosaccharide, more preferably all oligosaccharides in the mixture are mammalian milk oligosaccharides, most preferably all oligosaccharides in the mixture are human milk oligosaccharides.

In some embodiments, step i) comprises a first step of clarification by microfiltration. Alternatively, step i) comprises a first step of clarification by centrifugation, a first step of clarification by flocculation, or a first step of clarification by ultrafiltration.

In some embodiments, step i) comprises ultrafiltration.

Preferably, the ultrafiltration in step i) has a molecular weight cut-off equal to or higher than 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa. Alternatively or preferably, step i) comprises two consecutive ultrafiltrations, and wherein the membrane molecular weight cut-off of the first ultrafiltration is higher than that of the second ultrafiltration.

In a preferred embodiment, step ii) comprises nanofiltration and/or electrodialysis. Preferably, the nanofiltration and/or electrodialysis is performed twice. More preferably, the nanofiltration and/or electrodialysis steps are performed consecutively.

In some embodiments, the ultrafiltration permeate of step i) is nanofiltered and/or electrodialysed in step ii).

In an embodiment, the cationic ion exchanger treatment is a strongly acidic cation exchanger treatment, preferably treatment with a strong cation exchange resin in H+ form, K+ or Na+ form.

In some embodiments, step i) is ultrafiltration, and step ii) is nanofiltration and/or electrodialysis treatment combined with treatment with an ion exchange resin and/or chromatography. Preferably, the ion exchange resin is a strongly acidic cation exchange resin and/or a weakly basic anion exchange resin. More preferably, the ion exchange resin is a strongly acidic cation exchange resin and a weakly basic anion exchange resin.

In still another preferred embodiment of the method of this disclosure, step ii) comprises treatment with a strong cation exchange resin in H+ form and a weak anion exchange resin in free base form, preferably in Cl— form, alternatively preferably in OH— form. Preferably, the treatment with a strong cation exchange resin in H+-form is directly followed by a treatment with a weak anion exchange resin in free base form.

In a one preferred embodiment of the method of this disclosure, the method does not comprise electrodialysis. In some embodiments the method does comprise electrodialysis.

In an embodiment of this disclosure wherein the step i) is ultrafiltration, the step ii) is nanofiltration and/or electrodialysis treatment combined with treatment with an ion exchange resin being strongly acidic cation exchange resin and/or a weakly basic anion exchange resin, the treatment with a strong cation exchange resin and/or a weak anion exchange resin is preceded by ultrafiltration followed by nanofiltration and/or electrodialysis.

In a specific embodiment of this disclosure, one or more steps i) to iii) are performed more than once.

In some embodiments using ultrafiltration in step i) and nanofiltration in step ii), preferably the nanofiltration membrane has a molecular weight cut-off that is lower than that of the ultrafiltration membrane in step i).

As used herein, the molecular weight cut-off of the nanofiltration membrane in step ii) is preferably equal to or higher than 200 Da. Such as 200 Da, 300 Da, 400 Da, 500 Da, 600 Da, 700 Da, 800 Da, 900 Da, or 1000 Da. Preferably, between 300 and 500 Da and/or between 600 and 800 Da.

As used herein, when step ii) comprises an ion exchange resin treatment and/or chromatography, the ion exchange resin or chromatography is preferably on a neutral solid phase.

The method of this disclosure provides preferably a solution comprising the purified mixture of oligosaccharides with a Brix value of from about 8% to about 75%, preferably the solution comprising the purified mixture of oligosaccharides has a Brix value of from about 30% to about 65%.

In an embodiment, the solution comprising the purified mixture of oligosaccharides contains at least the mixture of oligosaccharides in an amount totaling a saccharide amount of at least 20.0% (w/v), 30.0% (w/v), 35.0% (w/v), and up to 45.0% (w/v), 50.0% (w/v), 60.0% (w/v).

Another aspect of this disclosure provides for a method wherein the at least one cell is a fungal, yeast, bacterial, insect, animal, and plant and protozoan cell. Preferably, the cells used are cells of a micro-organism. Another aspect of this disclosure provides for a method wherein the at least one micro-organism is a fungal, yeast or bacterial cell as described herein. The at least one micro-organism is chosen from the list comprising a bacterium, a yeast, or a fungus. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, this disclosure specifically relates to a mutated and/or transformed Escherichia coli cell or strain as indicated above wherein the E. coli strain is a K12 strain. More preferably, the Escherichia coli K12 strain is E. coli MG1655.

The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae.

The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Candida, Hansenula, Kluyveromyces, Pichia, Schizosaccharomyces, Schwanniomyces, Torulaspora, Yarrowia, and Zygosaccharomyces; preferably selected from the group comprising: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii.

The latter fungus belongs preferably to the genus Rhizopus, Dictyostelium, Penicillium, Mucor or Aspergillus.

In an embodiment, the at least one micro-organism is an E. coli or yeast of lactose permease positive phenotype wherein the lactose permease is coded by the gene LacY or LAC12, respectively.

Further according to a preferred embodiment of this disclosure, step i) is preceded by an enzymatic treatment. Preferably, the enzymatic treatment comprises incubation of the cultivation or fermentation broth with one or more enzymes selected from the group comprising: glycosidase, lactase, b-galactosidase, fucosidase, sialidase, maltase, amylase, hexaminidase, glucuronidase, trehalase, and invertase. Preferably or alternatively, the enzymatic treatment converts lactose and/or sucrose to monosaccharides.

According to preferred embodiment, the method further comprises decolorization.

In an embodiment, the method further comprises a step of sterile filtration and/or endotoxin removal, preferably by filtration of the purified oligosaccharide mixture through a 3 kDa filter.

In an embodiment, the purified oligosaccharide mixture solution has an ash content below 1% (on total dry solid), preferably below 0.5% (on total dry solid). In an embodiment, the purified oligosaccharide mixture solution has a Lead content lower than 0.1 mg/kg dry solid, more preferably lower than 0.05 mg/kg dry solid, even more preferably below 0.02 mg/kg dry solid; Arsenic content lower than 0.2 mg/kg dry solid, more preferably lower than 0.1 mg/kg, even more preferably lower than 0.05 mg/kg dry solid, Cadmium content lower than 0.1 mg/kg dry solid, more preferably lower than 0.05 mg/kg dry solid, even more preferably below 0.02 mg/kg dry solid; and/or Mercury content lower than 0.5 mg/kg dry solid, more preferably lower than 0.2 mg/kg dry solid, even more preferably below 0.1 mg/kg.

In an embodiment, the purified oligosaccharide mixture solution has a protein content below 100 mg per kg dry solid, DNA content below 10 ng per gram dry solid and/or endotoxin content below 10000 EU per gram dry solid. A protein content below 100 mg per kg dry solid is preferably below 100 mg, below 90 mg, below 80 mg, below 70 mg, below 60 mg, below 50 mg, below 40 mg, below 30 mg, below 20 mg, below 10 mg, below 5 mg per kg dry solid. A DNA content below 10 ng per gram dry solid is preferably below 10 ng, below 9 ng, below 8 ng, below 7 ng, below 6 ng, below 5 ng, below 4 ng, below 3 ng, below 2 ng, below 1 ng per gram dry solid. An endotoxin content below 10000 EU per gram dry solid is preferably below 7500 EU, below 5000 EU, below 2500 EU, below 1000 EU, below 750 EU, below 500 EU, below 250 EU, below 100 EU, below 50 EU per gram dry solid.

In some embodiments according to this disclosure, the purified oligosaccharide mixture solution is further concentrated to a syrup of at least 40% dry matter or the oligosaccharide mixture solution is dried to a powder. Preferably, the purified oligosaccharide mixture solution is dried. Such a step of drying can comprise any one or more of spray drying, lyophilization, evaporation, precipitation, spray freeze drying, freeze spray drying, band drying, belt drying, vacuum band drying, vacuum belt drying, drum drying, vacuum drum drying, roller drying, vacuum roller drying and other types of drying.

In some embodiments, the oligosaccharide mixture solution has a Brix value of from about 8 to about 75 percent Brix before drying, e.g., spray drying or lyophilization. In some embodiments, the oligosaccharide mixture solution has a Brix value of from about 30 to about 65 percent Brix before drying. In some embodiments, the oligosaccharide mixture solution has a Brix value of from about 50 to about 60 percent Brix before drying, preferably spray drying. In some embodiments, the oligosaccharide mixture solution has a Brix value of about 50 percent Brix before drying. Preferably, the purified oligosaccharide mixture solution is spray-dried. Alternatively or preferably, the step of drying is spray-drying or freeze-drying the purified oligosaccharide mixture solution and preferably wherein the pH of the solution is lower than 5.0.

In some embodiments, the purified oligosaccharide mixture is spray dried.

In some embodiments, the purified oligosaccharide mixture feed into the spray dryer has a Brix value of from about 8 to about 75 percent Brix. In some embodiments, purified oligosaccharide mixture feed into the spray dryer has a Brix value from about 30 to about 65 percent Brix. In some embodiments, purified oligosaccharide mixture feed into the spray dryer has a Brix value from about 50 to about 60 percent Brix.

In some embodiments, the feed into the spray dryer is at a temperature of from about 2 to about 70 degrees centigrade immediately before being dispersed into droplets in the spray dryer. In some embodiments, the feed into the spray dryer is at a temperature of from about 30 to about 60 degrees centigrade immediately before being dispersed into droplets in the spray dryer. In some embodiments, the feed into the spray dryer is at a temperature of from about 2 to about 30 degrees centigrade immediately before being dispersed into droplets in the spray dryer. In some embodiments, the spray drying uses air having an air inlet temperature of from 120 to 280 degrees centigrade In some embodiments, the air inlet temperature is from 120 to 210 degrees centigrade In some embodiments, the air inlet temperature is from about 130 to about 190 degrees centigrade In some embodiments, the air inlet temperature is from about 135 to about 160 degrees centigrade In some embodiments, the spray drying uses air having an air outlet temperature of from about 80 to about 110 degrees centigrade In some embodiments, the air outlet temperature is from about 100 to about 110 degrees centigrade In some embodiments, the spray drying is carried out at a temperature of from about 20 to about 90 degrees centigrade In some embodiments, the spray dryer is a co-current spray dryer. In some embodiments, the spray dryer is attached to an external fluid bed. In some embodiments, the spray dryer comprises a rotary disk, a high pressure nozzle or a two-fluid nozzle. In some embodiments, the spray dryer comprises an atomizer wheel. In some embodiments, spray-drying is the final purification step for the mixture of oligosaccharides.

In an embodiment, lyophilization is the final purification step for the mixture of oligosaccharides.

In an embodiment, a syrup is the final product for the mixture of oligosaccharides.

Oligosaccharide Mixture Powder

This specification provides an oligosaccharide mixture powder prepared by the process disclosed in this specification.

In some embodiments, the oligosaccharide mixture powder comprises a mixture of oligosaccharides as described herein.

In an embodiment, a spray dried powder essentially comprising or containing a mixture of structurally distinct oligosaccharides is provided, preferably for the production of a nutritional composition, a dietary supplement, a pharmaceutical ingredient, and/or a cosmetics ingredient.

In one embodiment, the dried powder contains a low amount of water.

Techniques for measuring moisture content of a material are well known in the art. Examples include Karl-Fischer titration, wherein the quantity of Karl-Fischer solution absorbed by a sample indicates the amount of water in the sample, and gravimetric methods, wherein a sample is dried and weight loss due to evaporation of solvent is measured at intervals.

In an embodiment, this disclosure provides the produced oligosaccharide mix that is dried to powder, wherein the dried powder contains≤15%-wt. of water, preferably ≤10%-wt. of water, more preferably ≤7%-wt. of water, most preferably ≤5%-wt. of water.

In an additional and/or alternative embodiment, the dried powder is free of genetically-engineered microorganisms and free of nucleic acid molecules derived from genetically-engineered microorganisms.

In a specific embodiment, this disclosure provides the produced oligosaccharide mix that is spray-dried to powder, wherein the spray-dried powder contains≤15%-wt. of water, preferably ≤10%-wt. of water, more preferably ≤7%-wt. of water, most preferably ≤5%-wt. of water. In some embodiments, the spray dryer is operated to achieve a moisture content of from about 3.0 to 5.0%-wt. of water. In some embodiments, the oligosaccharide mixture powder has a moisture content of less than 5%-wt. of water. In some embodiments, the oligosaccharide mixture powder has a moisture content of less than about 2.3 percent (by weight) water.

This specification provides an oligosaccharide mixture in powder form, wherein the powder has a mean particle size of 50 to 250 μm determined by laser diffraction, preferably the powder has a mean particle size of 95 to 120 μm determined by laser diffraction; more preferably the powder has a mean particle size of 110 to 120 μm. Such particle sizes are dependent on the dryer's specifications, configuration, performance and design. Commercial driers available are, for example, the Buchi mini spray dryer, Procept spray dryers, Gea spray dryers, optionally with either rotary atomizer, two-fluid nozzle, pressure nozzle, combi-nozzle, optionally in open-mode design, multi-stage drying design, closed-cycle design. The above particle sizes where obtained using Buchi spray dryer (Buchi Mini Spray Dryer B-290) (Büchi, Essen, Germany), applying the following parameters: Inlet temperature: 130° C., Outlet temperature 67° C.-71° C., gas flow 670 L/h, aspirator 100%.

In an embodiment, oligosaccharide mixture powder when re-dissolved in water at a concentration of 10% (mass on volume) provides a solution with a pH between 4 and 7, preferably with a pH between 4 and 6.

This specification also provides an oligosaccharide mixture in powder form, wherein the oligosaccharide mixture powder is having a loose bulk density of from about 500 g/L to about 700 g/L, a 100× tapped bulk density of from about 600 g/L to about 850 g/L, a 625× tapped bulk density of from about 600 g/L to about 900 g/L and/or a 1250× tapped bulk density of from about 650 g/L to about 900 g/L.

In some embodiments, the oligosaccharide mixture powder has a loose bulk density of from about 600 g/L to about 700 g/L. In some embodiments, the oligosaccharide mixture powder has a loose bulk density of from about 500 g/L to about 600 g/L.

In some embodiments, the oligosaccharide mixture powder has a 100× tapped bulk density of from about 750 g/L to about 850 g/L. In some embodiments, the oligosaccharide mixture powder has 100× tapped bulk density of from about 600 g/L to about 700 g/L.

In some embodiments, the oligosaccharide mixture powder has a 625× tapped bulk density of from about 750 g/L to about 900 g/L. In some embodiments, the oligosaccharide mixture powder has a 625× tapped bulk density of from about 700 g/L to about 800 g/L.

In some embodiments, the oligosaccharide mixture powder has a 1250× tapped bulk density of from about 850 g/L to about 900 g/L. In some embodiments, the oligosaccharide mixture powder has a 1250× tapped bulk density of from about 750 g/L to about 800 g/L.

In some embodiments, the oligosaccharide mixture powder has a loose bulk density of from about 600 g/L to about 700 g/L, a 100× tapped bulk density of from about 750 g/L to about 850 g/L, a 625× tapped bulk density of from about 750 g/L to about 850 g/L and/or a 1250× tapped bulk density of from about 850 g/L to about 900 g/L. In some embodiments, the oligosaccharide mixture powder has a loose bulk density of from about 500 g/L to about 600 g/L, a 100× tapped bulk density of from about 600 g/L to about 700 g/L, a 625× tapped bulk density of from about 700 g/L to about 800 g/L and/or a 1250× tapped bulk density of from about 750 g/L to about 800 g/L.

Oligosaccharide Mix Compositions

In an embodiment, the oligosaccharide mixture contains 2′FL, 3FL, and LDFT wherein the relative percentage of 2′FL to the sum of the masses of 2′FL, 3FL and LDFT is between 65% and 79%, the relative percentage of 3FL to the sum of the masses of 2′FL, 3FL and LDFT is between 17% and 21% and the relative percentage of LDFT to the sum of the masses of 2′FL, 3FL and LDFT is between 9% and 10%.

In an embodiment, the oligosaccharide mixture contains 2′FL, 3FL, and LDFT wherein the relative percentage of 2′FL to the sum of the masses of 2′FL, 3FL and LDFT is between 10% and 12%, the relative percentage of 3FL to the sum of the masses of 2′FL, 3FL and LDFT is between 79% and 96% and the relative percentage of LDFT to the sum of the masses of 2′FL, 3FL and LDFT is between 1% and 2%.

In an embodiment, the oligosaccharide mixture contains 2′FL, 3FL, LDFT, 3′SL and 6′SL wherein the relative percentage of 2′FL to the sum of the masses of 2′FL, 3FL, LDFT, 3′SL and 6′SL is between 53% and 64%, the relative percentage of 3FL to the sum of the masses of 2′FL, 3FL, LDFT, 3′SL and 6′SL is between 14% and 17% and the relative percentage of LDFT to the sum of the masses of 2′FL, 3FL, LDFT, 3′SL and 6′SL is between 7% and 8% and the relative percentage of 3′SL to the sum of the masses of 2′FL, 3FL, LDFT, 3′SL and 6′SL is between 5% and 6% and the relative percentage of 6′SL to the sum of the masses of 2′FL, 3FL, LDFT, 3′SL and 6′SL is between 12% and 15%.

In an embodiment, the oligosaccharide mixture contains LNT, LNnT, 3′SL and 6′SL wherein the relative percentage of LNT to the sum of the masses of LNT, LNnT, 3′SL and 6′SL is between 48% and 59%, the relative percentage of LNnT to the sum of the masses of LNT, LNnT, 3′SL and 6′SL is between 13% and 16% and the relative percentage of 3′SL to the sum of the masses of LNT, LNnT, 3′SL and 6′SL is between 8% and 10% and the relative percentage of 6′SL to the sum of the masses of LNT, LNnT, 3′SL and 6′SL is between 20% and 25%.

In an embodiment, the oligosaccharide mixture contains 3′SL and 6′SL wherein the relative percentage of 3′SL to the sum of the masses of 3′SL and 6′SL is between 26% and 32% and the relative percentage of 6′SL to the sum of the masses of 3′SL and 6′SL is between 64% and 78%.

In an embodiment, the oligosaccharide mixture contains 3′SL and 6′SL wherein the relative percentage of 6′SL to the sum of the masses of 3′SL and 6′SL is between 26% and 32% and the relative percentage of 3′SL to the sum of the masses of 3′SL and 6′SL is between 64% and 78%.

In an embodiment, the oligosaccharide mixture contains 2′FL, 3FL, LDFT, LNFP I, LNFP II, LNFP III and LNFP V wherein the relative percentage of 2′FL to the sum of the masses 2′FL, 3FL, LDFT, LNFP I, LNFP II, LNFP III and LNFP V is between 38% and 46%, the relative percentage of 3FL to the sum of the masses of 2′FL, 3FL, LDFT, LNFP I, LNFP II, LNFP III and LNFP V is between 10% and 12% and the relative percentage of LDFT to the sum of the masses of 2′FL, 3FL, LDFT, LNFP I, LNFP II, LNFP III and LNFP V is between 5% and 6% and the relative percentage of LNFP I to the sum of the masses of 2′FL, 3FL, LDFT, LNFP I, LNFP II, LNFP III and LNFP V is between 21% and 25% and the relative percentage of LNFP II to the sum of the masses of 2′FL, 3FL, LDFT, LNFP I, LNFP II, LNFP III and LNFP V is between 10% and 13%, and the relative percentage of LNFP III to the sum of the masses of 2′FL, 3FL, LDFT, LNFP I, LNFP II, LNFP III and LNFP V is between 5% and 6% and the relative percentage of LNFP V to the sum of the masses of 2′FL, 3FL, LDFT, LNFP I, LNFP II, LNFP III and LNFP V is between 1% and 2%.

In an embodiment, the oligosaccharide mixture contains 2′FL, 3FL, LDFT, LNFP I, LNFP II, LNFP III and LNFP V wherein the relative percentage of 2′FL to the sum of the masses 2′FL, 3FL, LDFT, LNFP I, LNFP II, LNFP III and LNFP V is between 6% and 8%, the relative percentage of 3FL to the sum of the masses of 2′FL, 3FL, LDFT, LNFP I, LNFP II, LNFP III and LNFP V is between 51% and 63% and the relative percentage of LDFT to the sum of the masses of 2′FL, 3FL, LDFT, LNFP I, LNFP II, LNFP III and LNFP V is between 0.5% and 2% and the relative percentage of LNFP I to the sum of the masses of 2′FL, 3FL, LDFT, LNFP I, LNFP II, LNFP III and LNFP V is between 3% and 5% and the relative percentage of LNFP II to the sum of the masses of 2′FL, 3FL, LDFT, LNFP I, LNFP II, LNFP III and LNFP V is between 17% and 21%, and the relative percentage of LNFP III to the sum of the masses of 2′FL, 3FL, LDFT, LNFP I, LNFP II, LNFP III and LNFP V is between 8% and 10% and the relative percentage of LNFP V to the sum of the masses of 2′FL, 3FL, LDFT, LNFP I, LNFP II, LNFP III and LNFP V is between 2% and 3%.

In an embodiment, the oligosaccharide mixture contains LSTa, LSTb, and LSTc wherein the relative percentage of LSTa to the sum of the masses of LSTa, LSTb, and LSTc is between 15% and 18% and the relative percentage of LSTb to the sum of the masses of LSTa, LSTb, and LSTc is between 13% and 16%, and the relative percentage of LSTc to the sum of the masses of LSTa, LSTb, and LSTc is between 62% and 75%

In an embodiment, the oligosaccharide mixture contains 3′SL, 6′SL, LSTa, LSTb, and LSTc wherein the relative percentage of 3′SL to the sum of the masses of 3′SL, 6′SL, LSTa, LSTb, and LSTc is between 14% and 17% and the relative percentage of 6′SL to the sum of the masses of 3′SL, 6′SL, LSTa, LSTb, and LSTc between 35% and 43% and the relative percentage of LSTa to the sum of the masses of 3′SL, 6′SL, LSTa, LSTb, and LSTc between 7% and 9% and the relative percentage of LSTb to the sum of the masses of 3′SL, 6′SL, LSTa, LSTb, and LSTc between 6% and 8% and the relative percentage of LSTc to the sum of the masses of 3′SL, 6′SL, LSTa, LSTb, and LSTc between 28% and 34%.

In an embodiment, the oligosaccharide mixture contains 2′FL, 3FL, LDFT, LNFP I, LNFP II, LNFP III, LNFP V, 3′SL, 6′SL, LSTa, LSTb, and LSTc wherein the relative percentage of 2′FL to the sum of the masses 2′FL, 3FL, LDFT, LNFP I, LNFP II, LNFP III, LNFP V, 3′SL, 6′SL, LSTa, LSTb, and LSTc is between 20% and 30%, and the relative percentage of 3FL to the sum of the masses 2′FL, 3FL, LDFT, LNFP I, LNFP II, LNFP III, LNFP V, 3′SL, 6′SL, LSTa, LSTb, and LSTc is between 5% and 10%, and the relative percentage of LDFT to the sum of the masses 2′FL, 3FL, LDFT, LNFP I, LNFP II, LNFP III, LNFP V, 3′SL, 6′SL, LSTa, LSTb, and LSTc is between 3% and 6%, and the relative percentage of 3′SL to the sum of the masses 2′FL, 3FL, LDFT, LNFP I, LNFP II, LNFP III, LNFP V, 3′SL, 6′SL, LSTa, LSTb, and LSTc is between 2% and 4%, and the relative percentage of 6′SL to the sum of the masses 2′FL, 3FL, LDFT, LNFP I, LNFP II, LNFP III, LNFP V, 3′SL, 6′SL, LSTa, LSTb, and LSTc is between 5% and 10%, and the relative percentage of LNT to the sum of the masses 2′FL, 3FL, LDFT, LNFP I, LNFP II, LNFP III, LNFP V, 3′SL, 6′SL, LSTa, LSTb, and LSTc is between 11% and 20%, and the relative percentage of LNnT to the sum of the masses 2′FL, 3FL, LDFT, LNFP I, LNFP II, LNFP III, LNFP V, 3′SL, 6′SL, LSTa, LSTb, and LSTc is between 2% and 4%, and the relative percentage of LNFP I to the sum of the masses 2′FL, 3FL, LDFT, LNFP I, LNFP II, LNFP III, LNFP V, 3′SL, 6′SL, LSTa, LSTb, and LSTc is between 12% and 20%, %, and the relative percentage of LNFP II to the sum of the masses 2′FL, 3FL, LDFT, LNFP I, LNFP II, LNFP III, LNFP V, 3′SL, 6′SL, LSTa, LSTb, and LSTc is between 5% and 10%, and the relative percentage of LNFP III to the sum of the masses 2′FL, 3FL, LDFT, LNFP I, LNFP II, LNFP III, LNFP V, 3′SL, 6′SL, LSTa, LSTb, and LSTc is between 3% and 6%, and the relative percentage of LNFP V to the sum of the masses 2′FL, 3FL, LDFT, LNFP I, LNFP II, LNFP III, LNFP V, 3′SL, 6′SL, LSTa, LSTb, and LSTc is between 0.5% and 2%, and the relative percentage of LSTa to the sum of the masses 2′FL, 3FL, LDFT, LNFP I, LNFP II, LNFP III, LNFP V, 3′SL, 6′SL, LSTa, LSTb, and LSTc is between 0.5% and 2%, and the relative percentage of LSTb to the sum of the masses 2′FL, 3FL, LDFT, LNFP I, LNFP II, LNFP III, LNFP V, 3′SL, 6′SL, LSTa, LSTb, and LSTc is between 0.5% and 2%, and the relative percentage of LSTc to the sum of the masses 2′FL, 3FL, LDFT, LNFP I, LNFP II, LNFP III, LNFP V, 3′SL, 6′SL, LSTa, LSTb, and LSTc is between 4% and 8%.

In an embodiment, the oligosaccharide mixture contains 2′FL, 3FL, LDFT, 3′SL, 6′SL, LNT and LNnT wherein the relative percentage of 2′FL to the sum of the masses of 2′FL, 3FL, LDFT, 3′SL, 6′SL, LNT and LNnT is between 37% and 46%, the relative percentage of 3FL to the sum of the masses of 2′FL, 3FL, LDFT, 3′SL, 6′SL, LNT and LNnT is between 10% and 12% and the relative percentage of LDFT to the sum of the masses of 2′FL, 3FL, LDFT, 3′SL, 6′SL, LNT and LNnT is between 4% and 8% and the relative percentage of 3′SL to the sum of the masses of 2′FL, 3FL, LDFT, 3′SL, 6′SL, LNT and LNnT is between 2% and 5% and the relative percentage of 6′SL to the sum of the masses of 2′FL, 3FL, LDFT, 3′SL, 6′SL, LNT and LNnT is between 8% and 10% and the relative percentage of LNT to the sum of the masses of 2′FL, 3FL, LDFT, 3′SL, 6′SL, LNT and LNnT is between 20% and 25% and the relative percentage of LNnT to the sum of the masses of 2′FL, 3FL, LDFT, 3′SL, 6′SL, LNT and LNnT is between 5% and 10%.

In an embodiment, the oligosaccharide mixture contains 2′FL, 3FL, LDFT, 3′SL, 6′SL, LNT and LNnT wherein the relative percentage of 2′FL to the sum of the masses of 2′FL, 3FL, LDFT, 3′SL, 6′SL, LNT and LNnT is between 3% and 6%, the relative percentage of 3FL to the sum of the masses of 2′FL, 3FL, LDFT, 3′SL, 6′SL, LNT and LNnT is between 35% and 46% and the relative percentage of LDFT to the sum of the masses of 2′FL, 3FL, LDFT, 3′SL, 6′SL, LNT and LNnT is between 0.5% and 2% and the relative percentage of 3′SL to the sum of the masses of 2′FL, 3FL, LDFT, 3′SL, 6′SL, LNT and LNnT is between 2% and 5% and the relative percentage of 6′SL to the sum of the masses of 2′FL, 3FL, LDFT, 3′SL, 6′SL, LNT and LNnT is between 8% and 15% and the relative percentage of LNT to the sum of the masses of 2′FL, 3FL, LDFT, 3′SL, 6′SL, LNT and LNnT is between 25% and 31% and the relative percentage of LNnT to the sum of the masses of 2′FL, 3FL, LDFT, 3′SL, 6′SL, LNT and LNnT is between 5% and 10%.

The oligosaccharide mixtures contains LNT and LNnT wherein the relative percentage of LNT to the sum of the masses of LNT and LNnT is between 70% and 90%, the relative percentage of LNnT to the sum of the masses of LNT and LNnT is between 10% and 30%

The oligosaccharide mixtures contains LNT and LNnT wherein the relative percentage of LNT to the sum of the masses of LNT and LNnT is between 10% and 30%, the relative percentage of LNnT to the sum of the masses of LNT and LNnT is between 70% and 90%

Products Comprising an Oligosaccharide Mixture

In some embodiments, an oligosaccharide mixture purified by a process of this specification is incorporated into nutritional formulations (such as food, drink or feed), food supplements, dietary supplement, digestive health functional foods or other consumable products, intended for use with infants, children, adults or seniors. Other applications comprise oligosaccharide mixture purified by a process of this specification incorporated into pharmaceutical ingredient, cosmetic ingredient or medicine. In some embodiments, the oligosaccharide mixture is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the oligosaccharide mixture purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis (e.g., BB12), B. longum and B. infantis (e.g., Bi-26, Bi-07, Bb-02, EVC001-ActiBif)), Streptococcus species (Streptococcus thermophilus (e.g., TH-4) and Saccharomyces boulardii. In some embodiments, an oligosaccharide mixture purified by a process of this specification is orally administered in combination with such microorganism.

Examples of further ingredients for dietary supplements include disaccharides (such as lactose), monosaccharides (such as glucose and galactose), thickeners (such as gum arabic), acidity regulators (such as trisodium citrate, phosphoric acid, sulphuric acid, acetic acid, lactic acid, citric acid, tartric acid, malic acid, succinic acid, fumaric acid or salts thereof), water, skimmed milk, and flavorings.

In some embodiments, the oligosaccharide mixture is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, an oligosaccharide mixture purified by a process in this specification is included in infant formula to provide nutritional benefits similar to those provided by the oligosaccharides in human breast milk. In some embodiments, the oligosaccharide mixture is mixed with one or more ingredients of the infant formula. Examples of infant formula ingredients include nonfat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, B6, B12, C and D), minerals (such as potassium citrate, calcium citrate, magnesium chloride, sodium chloride, sodium citrate and calcium phosphate) and possibly HMOs. Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise nonfat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the oligosaccharide mixture's concentration in the infant formula is approximately the same concentration as the oligosaccharide's concentration generally present in human breast milk. In some embodiments, the concentration of each of the single oligosaccharides in the mixture of oligosaccharides in the infant formula is approximately the same concentration as the concentration of that oligosaccharide generally present in human breast milk.

In some embodiments, the oligosaccharide mixture is incorporated into a feed preparation, wherein the feed is chosen from the list comprising pet food, animal milk replacer, veterinary product, post weaning feed, or creep feed.

The oligosaccharide mixture purified by a process of this specification can be added to a pharmaceutically acceptable carriers such as conventional additives, adjuvants, excipients and diluents (water, gelatine, talc, sugars, starch, gum arabic, vegetable gums, vegetable oils, polyalkylene glycols, flavoring agents, preservatives, stabilizers, emulsifying agents, lubricants, colorants, fillers, wetting agents, etc.). Suitable carriers are described in the most recent edition of Remington's Pharmaceutical Sciences, a standard reference text in the field. When the oligosaccharide mixture purified by a process of this specification is added to the pharmaceutically acceptable carriers, a dosage in the form of, for example, but not limited to tablets, powders, granules, suspensions, emulsions, infusions, capsules, injections, liquids, elixirs, extracts and tincture can be made. To the above formulas, if needed, probiotics, e.g., lacto bacteria, Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g., palm olein, sunflower oil, safflower oil) and vitamins and minerals essential in a daily diet can also be further added.

Pharmaceutical compositions comprising the oligosaccharide mixture purified by a process of this specification can be manufactured by means of any usual manner known in the art, e.g., described in the most recent edition of Remington's Pharmaceutical Sciences, a standard reference text in the field.

In an exemplary embodiment, the purification of the produced oligosaccharide mixture from the cultivation or fermentation broth, comprises the following steps in any order:
a) clarifying the cultivation or fermentation broth as described herein,
b) contacting the clarified cultivation or fermentation broth with a nanofiltration membrane with a molecular weight cut-off (MWCO) of 600-3500 Da ensuring the retention of produced oligosaccharide and allowing at least a part of the proteins, salts, by-products, color and other related impurities to pass,
c) conducting a diafiltration process on the retentate from step b), using the membrane, with an aqueous solution of an inorganic electrolyte, followed by optional diafiltration with pure water to remove excess of the electrolyte,
d) and collecting the retentate enriched in the oligosaccharide mixture in the form of a salt from the cation of the electrolyte, and
e) optionally drying, preferably spray drying.

In an alternative exemplary embodiment, the purification of the produced oligosaccharide mix can be made in a process, comprising the following steps in any order: subjecting the clarified cultivation or fermentation broth to two membrane filtration steps using different membranes, wherein—one membrane has a molecular weight cut-off of between about 300 Dalton to about 500 Dalton, and—the other membrane as a molecular weight cut-off of between about 600 Dalton to about 800 Dalton.

In an alternative exemplary embodiment, the purification of the oligosaccharide mixture can be made in a process, comprising the following steps in any order comprising the step of treating the clarified cultivation or fermentation broth with a strong cation exchange resin in H+-form and a weak anion exchange resin in free base form.

In an alternative exemplary embodiment, the purification of the produced mixture of oligosaccharides can be made in a process, comprising the following steps: the fermentation broth or cultivation comprising the produced mixture of oligosaccharides, biomass, medium components and contaminants, and preferably wherein the purity of the produced mixture of oligosaccharides in the fermentation broth or cultivation is <80 percent,
characterized in that the fermentation broth or cultivation is applied to the following purification steps:
   i) separation of biomass from the fermentation broth or cultivation,
   ii) cationic ion exchanger treatment for the removal of positively charged material,
   iii) anionic ion exchanger treatment for the removal of negatively charged material,
   iv) nanofiltration step and/or electrodialysis step,
wherein a purified solution comprising the produced oligosaccharide mixture at a combined purity of greater than or equal to 80 percent is provided. Optionally the purified solution is spray-dried. Alternatively, the purified solution is concentrated to a syrup preferably of at least 40% dry matter.

In an alternative exemplary embodiment, the purification of the produced oligosaccharide mixture can be made in a process, comprising the following steps in any order: enzymatic treatment of the cultivation; removal of the biomass from the cultivation; ultrafiltration; nanofiltration; and a column chromatography step. Preferably, such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

Method to Produce Purified Charged Oligosaccharide

In an embodiment, the present specification provides for a method to produce a purified charged oligosaccharide. In an embodiment, such charged oligosaccharide is part of a mixture of oligosaccharides to be purified together. In an embodiment, the charged oligosaccharide is purified from the cultivation or fermentation broth.

In an embodiment, a method to produce a purified charged oligosaccharide is provided the method comprising:
culturing at least one cell, preferably a cell of a micro-organism, that synthesizes a charged oligosaccharide in a suitable cultivation or fermentation medium to form a cultivation or fermentation broth,
purifying the charged oligosaccharide from the cultivation or fermentation broth by,
   i) clarifying the cultivation or fermentation broth and
   ii) removing salts and/or medium components from the clarified cultivation or fermentation broth and/or
   iii) concentrating the oligosaccharide mixture in the clarified cultivation or fermentation broth,
characterized in that step ii) comprises a treatment with a) mixed bed ion exchange resin or b) cation and anion exchange resin, thereby providing a solution comprising the purified charged oligosaccharide.

In an embodiment, the anion exchange resin used in any one of a) or b) has a moisture content of 30-48% and is preferably a gel type anion exchanger, preferably selected from the group comprising DOWEX™ 1-X8, XA4023, XA3112, DIAION™ SA20A, DIAION™ SA10A.

In an embodiment, the anion exchange resin is in OH— form.

In an embodiment, the mixed bed ion exchange resin is mixed bed column of DIAION™ SA20A and AMBERLITE™ FPC 22H mixed in a ratio 1.1:1 to 1.9:1.

In an embodiment, the mixed bed ion exchange resin is mixed bed column of DOWEX™ 1-X8 and AMBERLITE™ FPC 22H mixed in a ratio 1.1:1 to 1.9:1.

In an embodiment, the mixed bed ion exchange resin is mixed bed column of XA4023 and AMBERLITE™ FPC 22H mixed in a ratio 1.1:1 to 1.9:1.

In an embodiment, the mixed bed ion exchange resin is mixed bed column of XA3112 and AMBERLITE™ FPC 22H mixed in a ratio 1.1:1 to 1.9:1.

In an embodiment, the mixed bed ion exchange resin is mixed bed column of DIAION™ SA10A and AMBERLITE™ FPC 22H mixed in a ratio 1.1:1 to 1.9:1.

In an embodiment, the charged oligosaccharide is chosen from the list comprising a sialylated oligosaccharide, a sulphated chitosans, a deacetylated chitosan.

In an embodiment, the step i) is a step as described herein.

In an embodiment, the step ii) further comprises any one of step ii) as described herein.

In an embodiment, the step iii) is a step as described herein.

In an embodiment, the solution comprising the purified charged oligosaccharide is dried as described herein. In an embodiment, the step of drying comprises any one or more of spray drying, lyophilization, evaporation, precipitation and drying. In an embodiment, the solution comprising the purified charged oligosaccharide is spray-dried as described herein.

In an embodiment, the solution comprising the purified charged oligosaccharide is further concentrated to a syrup of at least 40% dry matter.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features that are still to be explained below can be used not only in the respectively specified combinations, but also in other combinations or on their own, without departing from the scope of this disclosure.

This disclosure relates to following specific embodiments:
1. Method to produce a purified mixture of different oligosaccharides comprising:
   culturing at least one cell, preferably a cell of a micro-organism, that synthesizes a mixture of different oligosaccharides in a suitable cultivation or fermentation medium to form a cultivation or fermentation broth,
   purifying the oligosaccharide mixture from the cultivation or fermentation broth by:
      i) clarifying the cultivation or fermentation broth, and
      ii) removing salts and/or medium components from the clarified cultivation or fermentation broth, and/or
      iii) concentrating the oligosaccharide mixture in the clarified cultivation or fermentation broth,
thereby providing a solution comprising the purified mixture of oligosaccharides.
2. Method according to embodiment 1, wherein the step iii) comes before step ii).
3. Method according to any one of embodiments 1 or 2, wherein the oligosaccharide mixture comprises at least 2 different oligosaccharides, preferably at least 3 different oligosaccharides, more preferably at least 4 different oligosaccharides, even more preferably at least 5 different oligosaccharides, most preferably at least 6 different oligosaccharides.
4. Method according to any one of embodiments 1 to 3, wherein the oligosaccharide mixture comprises at least 2 different oligosaccharides that differ in degree of polymerization, preferably the oligosaccharide mixture comprises at least 3 different oligosaccharides that differ in degree of polymerization, more preferably the oligosaccharide mixture comprises at least 4 different oligosaccharides that differ in degree of polymerization.
5. Method according to any one of embodiments 1 to 4, wherein the oligosaccharide mixture comprises at least one neutral and at least one charged oligosaccharide.
6. Method according to any one of embodiments 1 to 5, wherein the cultivation or fermentation broth comprises the mixture of oligosaccharides, biomass, medium components and contaminants.
7. Method according to any one of embodiments 1 to 6 wherein at least one of the cells has been genetically modified to produce at least one oligosaccharide, preferably the at least one cell has been genetically modified to produce at least two different oligosaccharides.
8. Method according to any one of embodiments 1 to 7 wherein at least one of the cells is the cell of a micro-organism that has been genetically modified to produce at least one oligosaccharide, preferably the at least one micro-organism has been genetically modified to produce at least two different oligosaccharides.
9. Method according to any one of embodiments 1 to 8 wherein the mixture of oligosaccharides in the cultivation or fermentation broth is obtained by culturing at least one genetically modified cell, preferably a cell of a microorganism, capable of producing the mixture of oligosaccharides, preferably from an internalized carbohydrate precursor.
10. Method according to any one of embodiments 1 to 9, wherein the at least one cell, preferably micro-organism, is cultured in a minimal salt medium with a carbon source on which the at least one cell, preferably micro-organism, grows.
11. Method according to embodiment 10, wherein the minimal salt medium contains sulphate, phosphate, chloride, ammonium, calcium ion, magnesium ion, sodium ion, potassium ion, iron ion, copper ion, zinc ion, manganese ion, cobalt ion, and/or selenium ion.
12. Method according to any one of embodiments 10 or 11, wherein the carbon source comprises one or more of glucose, fructose, mannose, sucrose, maltose, corn steep liquor, lactose, galactose, high fructose syrup, starch, cellulose, hemi-cellulose, malto-oligosaccharides, trehalose, glycerol, acetate, citrate, lactate and pyruvate.
13. Method according to any one of embodiments 1 to 12, wherein the combined purity of the mixture of oligosaccharides in the cultivation or fermentation broth is less than 80% on total dry solid, and/or wherein the combined purity of the purified mixture of oligosaccharides in the solution is equal to or more than 80% on total dry solid.
14. The method according to any one of steps 1 to 13, wherein the combined purity of the mixture of oligosaccharides in the cultivation or fermentation broth is <70%, <60%, <50%, <40%, <30%, <20%, <10% on total dry solid, before the purification and/or the combined purity of the purified mixture of oligosaccharides is >80%, preferably of >85%, more preferably >90%, even more preferably >95%, most preferably >97% on total dry solid after the purification.
15. Method according to any one of embodiments 1 to 14, wherein the step i) of clarifying the cultivation or fermentation broth comprises one or more of clarification, clearing, filtration, microfiltration, centrifugation, decantation and ultrafiltration, preferably the step i) further comprising use of a filter aid and/or flocculant; preferably the filtration aid is an adsorbing agent, more preferably active carbon.
16. Method according to embodiment 15, wherein the step i) comprises subjecting the cultivation or fermentation broth to two membrane filtration steps using different membranes.
17. Method/process according to any one of embodiments 1 to 16, wherein the step ii) of removing salts and/or medium components from the clarified cultivation or fermentation broth comprises at least one or more of nanofiltration, dialysis, electrodialysis, use of activated charcoal or carbon, use of solvents, use of alcohols, and use of aqueous alcohol mixtures, use of charcoal, tangential flow high-performance filtration, tangential flow ultrafiltration, affinity chromatography, ion exchange, cation exchange, anion exchange, mixed bed ion exchange, simulated moving bed chromatography, ion exchange chromatography, hydrophobic interaction chromatography, gel filtration, ligand exchange chromatography, column chromatography, cation exchange adsorbent resin, and use of ion exchange resin.
18. The method according to embodiment 17, wherein the step ii) of removing salts and/or medium components from the clarified cultivation or fermentation broth comprises anion exchange wherein the anion exchange resin is characterized to have a moisture content of 30-48% and preferably microporous or a gel type anion exchanger, preferably selected from the group DOWEX™ 1-X8, XA4023, XA3112, DIAION™ SA20A, DIAION™ SA10A.
19. The method according to any one of claims 1 to 18, wherein step ii) comprises a treatment with a mixed bed ion exchange resin, preferably mixed bed column of DIAION™ SA20A and AMBERLITE™ FPC 22H mixed in a ratio 1.1:1 to 1.9:1.
20. Method according to any one of embodiments 1 to 19, wherein the step iii) of concentrating comprises one or more of nanofiltration, reverse osmosis, evaporation, wiped film evaporation, and falling film evaporation.
21. Method according to any one of embodiments 1 to 20, wherein the mixture of oligosaccharides comprises at least one of a fucosylated oligosaccharide, sialylated oligosaccharide, Lewis type antigen, an N-acetylglucosamine containing neutral oligosaccharide, N-acetyllactosamine containing oligosaccharide, lacto-N-biose containing oligosaccharide, non-fucosylated neutral oligosaccharide, chitosan, chitosan oligosaccharide, heparosan, chondroitin sulphate, glycosaminoglycan oligosaccharide, heparin, heparan sulphate, chondroitin sulphate, dermatan sulphate, hyaluronan or hyaluronic acid and/or keratan sulphate.
22. Method according to any one of embodiments 1 to 21, wherein the mixture of oligosaccharides comprises at least one mammalian milk oligosaccharide, preferably at least one human milk oligosaccharide, more preferably all oligosaccharides in the mixture are mammalian milk oligosaccharides, most preferably all oligosaccharides in the mixture are human milk oligosaccharides.
23. Method according to any one of embodiments 1 to 22, wherein the step i) comprises a first step of clarification by microfiltration.
24. Method according to any one of embodiments 1 to 22, wherein the step i) comprises a first step of clarification by centrifugation.
25. Method according to any one of embodiments 1 to 22, wherein the step i) comprises a first step of clarification by flocculation.
26. Method according to any one of embodiments 1 to 22, wherein the step i) comprises a first step of clarification by ultrafiltration.
27. Method according to any one of embodiments 1 to 26, wherein the step i) comprises ultrafiltration.
28. The method according to any one of embodiments 26 or 27, wherein in step i) the ultrafiltration has a molecular weight cut-off equal to or higher than 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa.
29. The method according to any one of embodiments 1 to 28, wherein step i) comprises two consecutive ultrafiltrations, and wherein the membrane molecular weight cut-off of the first ultrafiltration is higher than that of the second ultrafiltration.
30. The method according to any one of embodiments 1 to 29, wherein step ii) comprises nanofiltration and/or electrodialysis.
31. The method according to embodiment 30, wherein the nanofiltration and/or electrodialysis is performed twice.
32. The method according to embodiment 31, wherein the nanofiltration and/or electrodialysis steps are performed consecutively.
33. The method according to any one of embodiments 26 to 32, wherein the ultrafiltration permeate of step i) is nanofiltered and/or electrodialysed in step ii).
34. Method according to any one of embodiments 1 to 22, 26 to 33 wherein the step i) is ultrafiltration, the step ii) is nanofiltration and/or electrodialysis treatment combined with treatment with an ion exchange resin and/or chromatography.
35. The method according to embodiment 34, wherein the ion exchange resin is a strongly acidic cation exchange resin and/or a weakly basic anion exchange resin.
36. The method according to embodiment 35, wherein the ion exchange resin is a strongly acidic cation exchange resin and a weakly basic anion exchange resin.
37. Method according to any one of embodiments 1 to 36, wherein the step ii) comprises treatment with a strong cation exchange resin in H+ form or Na+ form and a weak anion exchange resin in free base form, preferably in Cl— form, alternatively preferably in OH-form.
38. Method according to any one of embodiments 35 to 37, wherein the treatment with a strong cation exchange resin in H+-form or Na+ form is directly followed by a treatment with a weak anion exchange resin in free base form.
39. Method according to any one of embodiments 1 to 38, which does not comprise electrodialysis.
40. Method according to any one of embodiments 1 to 38, wherein step ii) comprises electrodialysis.
41. Method according to any one of embodiments 35 to 38 or 40, wherein the treatment with a strong cation exchange resin and/or a weak anion exchange resin is preceded by ultrafiltration followed by nanofiltration and/or electrodialysis.
42. Method according to any one of embodiments 1 to 41, wherein any one or more of the steps i) to iii) is performed more than once.
43. Method according to any one of embodiments 33 to 42, wherein the molecular weight cut-off of the nanofiltration membrane in step ii) is lower than that of the ultrafiltration membrane in step i).
44. Method according to any of embodiments 33 to 43, wherein the molecular weight cut-off of the nanofiltration membrane in step ii) is equal or higher than 200 Da, 300 Da, 400 Da, 500 Da, 600 Da, 700 Da, 800 Da, 900 Da, or 1000 Da.
45. Method according to any of the embodiments 17 to 44, wherein step ii) comprises an ion exchange resin treatment and/or chromatography on a neutral solid phase.
46. Method according to any one of embodiments 1 to 45, wherein the solution comprising the purified mixture of oligosaccharides has a Brix value of from about 8 to about 75%, preferably the solution comprising the purified mixture of oligosaccharides has a Brix value of from about 30 to about 65%.
47. Method according to any one of embodiments 1 to 46, wherein the at least one micro-organism is an E. coli or yeast of lactose permease positive phenotype wherein the lactose permease is coded by the gene LacY or LAC12, respectively.
48. Method according to any one of embodiments 1 to 47, wherein the micro-organism is a bacterium, preferably an Escherichia coli strain, more preferably an Escherichia coli strain that is a K-12 strain, even more preferably the Escherichia coli K-12 strain is E. coli MG1655.
49. Method according to any one of embodiments 1 to 47, wherein the at least one micro-organism is a yeast.
50. Method of embodiment 49, wherein the yeast is selected from the group comprising: Saccharomyces, Candida, Hansenula, Kluyveromyces, Pichia, Schizosaccharomyces, Schwanniomyces, Torulaspora, Yarrowia, and Zygosaccharomyces; preferably selected from the group comprising: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii.
51. Method according to any one of embodiments 1 to 50 wherein step i) is preceded by an enzymatic treatment.
52. Method according to embodiment 51, wherein the enzymatic treatment comprises incubation of the cultivation or fermentation broth with one or more enzymes selected from the group comprising: glycosidase, lactase, b-galactosidase, fucosidase, sialidase, maltase, amylase, hexaminidase, glucuronidase, trehalase, and invertase.
53. Method according to any one of embodiments 51 or 52, wherein the enzymatic treatment converts lactose and/or sucrose to monosaccharides.
54. Method of any one of embodiments 1 to 53, wherein the method further comprises decolorization.
55. Method according to any one of embodiments 1 to 54, wherein the purified oligosaccharide mixture solution has an ash content below 1% (on total dry solid), preferably below 0.5% (on total dry solid), preferably with Lead content lower than 0.1 mg/kg dry solid, Arsenic content lower than 0.2 mg/kg dry solid, Cadmium content lower than 0.1 mg/kg dry solid and/or Mercury content was lower than 0.5 mg/kg dry solid.
56. Method according to any one of embodiments 1 to 55, wherein the purified oligosaccharide mixture solution is further concentrated to a syrup of at least 40% dry matter or the oligosaccharide mixture solution is dried to a powder.
57. Method according to any one of embodiments 1 to 56, wherein the purified oligosaccharide mixture solution has a protein content below 100 mg per kg dry solid, DNA content below 10 ng per gram dry solid and/or endotoxin content below 10000 EU per gram dry solid.
58. Method of any one of embodiments 1 to 57, wherein the purified oligosaccharide mixture solution is dried.
59. Method according to any one of embodiments 57 or 58, wherein the step of drying comprises any one or more of spray drying, lyophilization, evaporation, precipitation and drying.
60. Method according to embodiment 59, wherein the purified oligosaccharide mixture solution is spray-dried.
61. Method according to embodiment 59, wherein the drying is spray-drying or freeze-drying the purified oligosaccharide mixture solution and preferably wherein the pH of the solution is lower than 5.0.
62. The purified mixture of oligosaccharides obtained according to the method according to any of embodiment 1 to 61.
63. The dried powder obtained according to any one of the method of embodiment 58 to 61, wherein the dried powder contains≤15%-wt. of water, preferably ≤10%-wt. of water, more preferably ≤7%-wt. of water, most preferably ≤5%-wt. of water.
64. The spray dried powder obtained according to any one of the method of embodiment 59 to 63, wherein the powder has a mean particle size of 50 to 250 μm, determined by laser diffraction; preferably the powder has a mean particle size of 95 to 120 μm, more preferably the powder has a mean particle size of 110 to 120 μm.

65. Oligosaccharide mix powder according to any one of claim 58 to 64 wherein the powder when redissolved in water at a concentration of 10% (mass on volume) provides a solution with a pH between 4 and 7, preferably with a pH between 4 and 6.
66. The dried purified mixture of oligosaccharides powder obtainable according to the method according to any one of embodiment 58 to 65, wherein the powder exhibits:
   a loose bulk density of from about 500 g/L to 700 g/L,
   a 100× tapped bulk density of from about 600 g/L to about 850 g/L,
   a 625× tapped bulk density of from about 600 g/L to about 900 g/L, and/or
   a 1250× tapped bulk density of from about 650 g/L to about 900 g/L.
67. The oligosaccharide mix powder obtained according to 66, wherein the powder exhibits:
   a loose bulk density of from about 600 g/L to 700 g/L,
   a 100× tapped bulk density of from about 750 g/L to about 850 g/L,
   a 625× tapped bulk density of from about 750 g/L to about 850 g/L, and/or
   a 1250× tapped bulk density of from about 850 g/L to about 900 g/L.
68. The oligosaccharide mix powder obtained according to 66, wherein the powder exhibits:
   a loose bulk density of from about 500 g/L to 600 g/L,
   a 100× tapped bulk density of from about 600 g/L to about 700 g/L,
   a 625× tapped bulk density of from about 700 g/L to about 800 g/L, and/or
   a 1250× tapped bulk density of from about 750 g/L to about 800 g/L.
69. Use of the purified mixture of oligosaccharides obtained according to the method of any one of embodiment 1 to 61 in a food or feed preparation, in a dietary supplement, in a cosmetic ingredient or in a pharmaceutical ingredient.
70. Use of the purified mixture of oligosaccharides according to any one of embodiment 62 to 68 in a food or feed preparation, in a dietary supplement, in a cosmetic ingredient or in a pharmaceutical ingredient.
71. The use according to any one of embodiments 69 or 70, wherein the food is a human food.
72. The use according to any one of embodiments 69 to 71, wherein the food is an infant food.
73. The use according to any one of embodiments 69 to 72, wherein the food is an infant formula or an infant supplement.
74. The use according to any one of embodiments 69 or 70 wherein the feed is a pet food, animal milk replacer, veterinary product, post weaning feed, or creep feed.
75. Method to produce a purified charged oligosaccharide comprising:
   culturing at least one cell, preferably a cell of a micro-organism, that synthesizes a charged oligosaccharide in a suitable cultivation or fermentation medium to form a cultivation or fermentation broth,
   purifying the charged oligosaccharide from the cultivation or fermentation broth by:
      i) clarifying the cultivation or fermentation broth, and
      ii) removing salts and/or medium components from the clarified cultivation or fermentation broth, and/or
      iii) concentrating the oligosaccharide mixture in the clarified cultivation or fermentation broth,
characterized in that step ii) comprises a treatment with a) a mixed bed ion exchange, or b) cation and anion exchange, thereby providing a solution comprising the purified charged oligosaccharide.
76. Method according to embodiment 75 wherein the anion exchange resin used in any one of a) or b) has a moisture content of 30-48% and is preferably a gel type anion exchanger, preferably selected from the group comprising Dowex 1-X8, XA4023, XA3112, DIAION™ SA20A, DIAION™ SA10A.
77. Method according to embodiment 76 wherein the anion exchange resin is in OH— form.
78. Method according to any one of embodiments 75 or 76 wherein the treatment is a) mixed bed ion exchange and the mixed bed ion exchange resin is mixed bed column of DIAION™ SA20A and AMBERLITE™ FPC 22H mixed in a ratio 1.1:1 to 1.9:1.
79. Method according to any one of embodiments 75 to 78, wherein the charged oligosaccharide is chosen from the list comprising a sialylated oligosaccharide, a sulphated chitosans, a deacetylated chitosan.
80. Method according to any one of embodiments 75 to 79, wherein the purified charged oligosaccharide solution is further concentrated to a syrup of at least 40% dry matter or the oligosaccharide mixture solution is dried to a powder.

This disclosure will be described in more detail in the examples.

### EXAMPLES

### Example 1. Materials and Methods

Media and Cultivation

The Luria Broth (LB) medium comprised 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄.7H₂O, 30 g/L sucrose or 30 g/L glycerol, 1 ml/L vitamin solution, 100 μL molybdate solution, and 1 mL/L selenium solution. As specified in the respective examples, 0.30 g/L sialic acid, 20 g/L lactose, 20 g/L LacNAc, 20 g/L LNnT, 20 g/L LNT and/or 20 g/L LNB were additionally added to the medium as precursor(s). The minimal medium was set to a pH of 7 with 1M KOH. Vitamin solution consisted of 3.6 g/L FeCl₂.4H₂O, 5 g/L CaCl₂.2H₂O, 1.3 g/L MnCl₂.2H₂O, 0.38 g/L CuCl₂.2H₂O, 0.5 g/L CoCl₂.6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L SeO₂.

The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 20 g/L lactose, 20 g/L LacNAc, 20 g/L LNnT, 20 g/L LNT and/or 20 g/L LNB were additionally added to the medium as precursor(s).

Complex medium was sterilized by autoclaving (121° C., 21 min) and minimal medium by filtration (0.22 μm Sartorius).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 or 30 L L bioreactor was then inoculated (250 mL inoculum in 2 L batch medium or 1 L in 17 L batch medium); the process was controlled by MFCS control software (Sartorius Stedim Biotech, Melsungen, Germany). Culturing conditions were set to 37° C., and maximal stirring; pressure gas flow rates were dependent on the strain and bioreactor. The pH was controlled at 6.8 using 0.5 M H2SO₄ and 20% NH₄OH. The exhaust gas was cooled. 10% solution of silicone antifoaming agent was added when foaming raised during the fermentation.

Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an OD₆₀₀ nm of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a GENE PULSER™ (BioRad) (600 Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with DpnIl re-purified from an agarose gel, and suspended in elution buffer (5 mM Tris, pH 8.0). Selected mutants were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock-ins are checked with control primers.

In one example for GDP-fucose and fucosylated oligosaccharide production, the mutant strain was derived from E. coli K₁₂ MG1655 comprising knock-outs of the E. coli wcaJ and thyA genes and genomic knock-ins of constitutive expression constructs containing a sucrose transporter like e.g., CscB originating from E. coli W (UniProt ID EOIXR1), a fructose kinase like e.g., frk originating from Zymomonas mobilis (ZmFrk) (UniProt ID Q03417), a sucrose phosphorylase like e.g., BaSP originating from Bifidobacterium adolescentis (UniProt ID A0ZZH6), additionally comprising expression plasmids with constitutive expression constructs for an alpha-1,2-fucosyltransferase like e.g., HpFutC from H. pylori (GenBank No. AAD29863.1) and/or an alpha-1,3-fucosyltransferase like e.g., HpFucT from H. pylori (UniProt ID O30511) and with a constitutive expression construct for the E. coli thyA (UniProt ID P0A884) as selective marker. The constitutive expression constructs of the fucosyltransferase genes can also be present in the mutant E. coli strain via genomic knock-ins. GDP-fucose production can further be optimized in the mutant E. coli strain by genomic knock-outs of the E. coli genes comprising glgC, agp, pfkA, pfkB, pgi, arcA, iclR, pgi and Ion as described in WO 2016075243 and WO 2012007481. GDP-fucose production can additionally be optimized comprising genomic knock-ins of constitutive expression constructs for a mannose-6-phosphate isomerases like e.g., manA from E. coli (UniProt ID P00946), a phosphomannomutase like e.g., manB from E. coli (UniProt ID P24175), a mannose-1-phosphate guanylyltransferase like e.g., manC from E. coli (UniProt ID P24174), a GDP-mannose 4,6-dehydratase like e.g., gmd from E. coli (UniProt ID POAC88) and a GDP-L-fucose synthase like e.g., fcl from E. coli (UniProt ID P32055). GDP-fucose production can also be obtained by genomic knock-outs of the E. coli fucK and fucI genes together with genomic knock-ins of constitutive expression constructs containing fucose permease like e.g., fucP from E. coli (UniProt ID P11551) and a bifunctional enzyme with fucose kinase/fucose-1-phosphate guanylyltransferase activity like e.g., fkp from Bacteroidesfragilis (UniProt ID SUV40286.1). If the mutant strain producing GDP-fucose was intended to make fucosylated lactose structures, the strain was additionally modified with genomic knock-outs of the E. coli LacZ, LacY and LacA genes and with a genomic knock-in of a constitutive expression construct for a lactose permease like e.g., the E. coli LacY (UniProt ID P02920).

In an example to produce lacto-N-triose (LN3, GlcNAc-b1,3-Gal-b1,4-Glc), the mutant strain was derived from E. coli K₁₂ MG1655 and modified with a knock-out of the E. coli lacZ, lacY, lacA and nagB genes and with genomic knock-ins of constitutive transcriptional units for a lactose permease like e.g., the E. coli LacY (UniProt ID P02920) and a galactoside beta-1,3-N-acetylglucosaminyltransferase like e.g., lgtA (UniProt ID Q9JXQ6) from N. meningitidis.

In an example for production of LN3 derived oligosaccharides like lacto-N-tetraose (LNT, Gal-b1,3-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,3-galactosyltransferase like e.g., wbgO from E. coli O55:H7.

In an example for production of LN3 derived oligosaccharides like lacto-N-neotetraose (LNnT, Gal-b1,4-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,4-galactosyltransferase like e.g., lgtB from Neisseria meningitidis.

Optical Density

Cell density of the cultures was frequently monitored by measuring optical density at 600 nm (Implen Nanophotometer NP80, Westburg, Belgium or with a Spark 10M microplate reader, Tecan, Switzerland).

Analytical Analysis

Standards such as, but not limited to, sucrose, lactose, N-acetyllactosamine (LacNAc, Gal-b1,4-GlcNAc), lacto-N-biose (LNB, Gal-b1,3-GlcNAc), fucosylated LacNAc (2′FLacNAc, 3-FLacNAc), sialylated LacNAc, (3′SLacNAc, 6′SLacNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase comprised a ¼ water and ¾ acetonitrile solution to which 0.2% triethylamine was added. The method was isocratic with a flow of 0.130 mL/min. The ELS detector had a drift tube temperature of 50° C. and the N₂ gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase comprised a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase comprised a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionization (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2% to 12% of eluent B over 21 min, a second increase from 12% to 40% of eluent B over 11 min and a third increase from 40% to 100% of eluent B over 5 min. As a washing step 100% of eluent B was used for 5 min. For column equilibration, the initial condition of 2% of eluent B was restored in 1 min and maintained for 12 min.

For identification of the single oligosaccharides in the mixture of oligosaccharides produced as described herein, the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the saccharide methods such as, e.g., acid-catalyzed hydrolysis, HPLC (high performance liquid chromatography) or GLC (gas-liquid chromatography) (after conversion to alditol acetates) may be used. To determine the glycosidic linkages, the saccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the oligosaccharide sequence, a partial depolymerization is carried out using an acid or enzymes to determine the structures. To identify the anomeric configuration, the oligosaccharide is subjected to enzymatic analysis, e.g., it is contacted with an enzyme that is specific for a particular type of linkage, e.g., beta-galactosidase, or alpha-glucosidase, etc., and NMR may be used to analyze the products.

Ash Content

The ash content is a measure of the total amount of minerals present within a food or ingredients such as oligosaccharides, whereas the mineral content is a measure of the amount of specific inorganic components present within a food, such as Ca, Na, K, Mg, phosphate, sulphate and Cl. Determination of the ash and mineral content of foods or oligosaccharides is important for a number of reasons: Nutritional labeling. The concentration and type of minerals present must often be stipulated on the label of a food or ingredient such as oligosaccharides. The quality of many foods depends on the concentration and type of minerals they contain, including their taste, appearance, texture and stability. Microbiological stability. High mineral contents are sometimes used to retard the growth of certain microorganisms. Nutrition. Some minerals are essential to a healthy diet (e.g., calcium, phosphorous, potassium and sodium) whereas others can be toxic (e.g., lead, mercury, cadmium and aluminum). Processing. It is often important to know the mineral content of foods/products during processing because this affects the physicochemical properties of foods or ingredient such as oligosaccharides.

Ash is the inorganic residue remaining after the water and organic matter have been removed by heating in the presence of oxidizing agents, which provides a measure of the total amount of minerals within a food. Analytical techniques for providing information about the total mineral content are based on the fact that the minerals (the analyte) can be distinguished from all the other components (the matrix) within a food or ingredient in some measurable way. The most widely used methods are based on the fact that minerals are not destroyed by heating, and that they have a low volatility compared to other food components. The three main types of analytical procedure used to determine the ash content of foods are based on this principle: dry ashing, wet ashing and low temperature plasma dry ashing. The method chosen for a particular analysis depends on the reason for carrying out the analysis, the type of food or ingredient analyzed and the equipment available. Ashing may also be used as the first step in preparing samples for analysis of specific minerals, by atomic spectroscopy or the various traditional methods described below.

For the sample preparation a sample whose composition represents that of the ingredient is selected to ensure that its composition does not change significantly prior to analysis. For instance, a dry oligosaccharide sample is generally hygroscopic and the selected sample should be kept under dry conditions avoiding the absorption of water. Typically, samples of 1-10 g are used in the analysis of ash content. Solid ingredients are finely ground and then carefully mixed to facilitate the choice of a representative sample. Before carrying out an ash analysis, samples that are high in moisture or in solution are generally dried to prevent spattering during ashing. Other possible problems include contamination of samples by minerals in grinders, glassware or crucibles that come into contact with the sample during the analysis. For the same reason, deionized water is used when preparing samples and the same is used in the blank sample.

Dry ashing procedures use a high temperature muffle furnace capable of maintaining temperatures of between 500 and 600° C. Water and other volatile materials are vaporized and organic substances are burned in the presence of the oxygen in air to CO₂, H₂O and N₂. Most minerals are converted to oxides, sulphates, phosphates, chlorides or silicates. Although most minerals have fairly low volatility at these high temperatures, some are volatile and may be partially lost, e.g., iron, lead and mercury, for these minerals ICP-MS analysis of the product is more appropriate for quantification.

The food sample is weighed before and after ashing to determine the concentration of ash present. The ash content can be expressed on dry basis is calculated by dividing the mass of the ashed material, ingredient, or food by the mass of the dry material, ingredient, or food before ashing. Multiplied with 100, this gives the percentage of ash in the material, ingredient, or food. In a similar way the wet ash percentage can be determined for liquid products, wherein the mass of the liquid before and after ashing is used instead of the mass of the dry material, ingredient, or food.

Heavy Metal Determination

A robust general inductively coupled plasma-mass spectrometry (ICP-MS) based method was used for the detection and quantitation for each of the following elements: arsenic (As), selenium (Se), cadmium (Cd), tin (Sn), lead (Pb), silver (Ag), palladium (Pd), platinum (Pt), mercury (Hg), molybdenum (Mo), sodium (Na), potassium (K), Calcium (Ca), Magnesium (Mg), Iron (Fe), zinc (Zn), manganese (Mn), Phosphorus (P), selenium (Se).

Nitric acid (>65%, Sigma-Aldrich) was used for microwave digestion and standard/sample preparation. All dilutions were done using 18.2 MQ cm (Millipore, Bedford, MA, USA) de-ionized water (DIW). About 0.2 g of each oligosaccharide, ingredient, sample were digested in 5 mL of HNO₃ using the microwave digestion (CEM, Mars 6) program 15 minutes (min) ramping time and 15 min holding time at 100 W and 50° C. followed by 15 min ramping time and 20 min holding time at 1800 W and 210° C. The samples were cooled after digestion for 30 minutes. 1. The fully digested samples were then diluted to 50 mL with DIW.

Analyses were carried out using a standard Agilent 7800 ICP-MS, which includes the fourth-generation ORS cell system for effective control of polyatomic interferences using helium collision mode (He mode). The ORS controls polyatomic interferences using He to reduce the transmission of all common matrix-based polyatomic interferences. Smaller, faster analyte ions are separated from larger, slower interference-ions using kinetic energy discrimination (KED). All elements, except Se, were measured in He mode with a flow rate of 5 mL/min. Se was measured in High Energy He (HEHe) mode, using a cell gas flow rate of 10 mL/min. The 7800 ICP-MS was configured with the standard sample introduction system comprised a MicroMist glass concentric nebulizer, quartz spray chamber, quartz torch with 2.5 mm i.d. injector, and nickel interface cones. The ICP-MS operating conditions are: 1550 W RF power, 8 mm sampling depth, 1.16 l/min nebulizing gas, autotuned lens tuning, 5 or 10 ml/min helium gas flow, 5 V KED.

Dry Matter/Dry Solid and Moisture Content Quantification

Sartorius MA150 Infrared Moisture Analyzer is used to determine the dry matter content of the oligosaccharides. 0.5 g of oligosaccharide is weighed on an analytical balance and is dried in the infrared moisture analyzer until the weight of the sample is stable. The mass of the dried sample divided by the mass of the sample before drying gives the dry matter content (in percent) of the oligosaccharides or sample including oligosaccharides. In a similar way a liquid sample is weighed, however, the amount of liquid weighed is adapted to the expected amount of dry matter in the liquid, so the mass of the dry matter is properly measurable on an analytical balance.

A moisture analyzer measures the dry matter, but not the water content. Karl Fisher titration is used to determine the amount of water present in a powder, ingredient of food. The KF titration is carried out with a Karl Fischer titrator DL31 from Mettler Toledo using the two-component technique with Hydra-Point Solvent G and Hydra-Point titrant (5 mg H₂O/ml), both purchased from J. T. Baker (Deventer, Holland). The polarizing current for bipotentiometric end-point determination was 20 microA and the stop voltage 100 mV. The end-point criterion was the drift stabilization (15 micro gram H₂O min⁻¹) or maximum titration time (10 min).

The moisture content (MC) of sample was calculated using the following equation:

MC=V_KF W_eq100/W_sample; where V_KF is the consumption of titrant in mL, W_eq the titer of titrant in mg H₂O/mL and W_sample the weight of sample in mg.

Biomass Dry Mass Content (Cell Dry Mass)

Cell dry weight was obtained by centrifugation (15 min, 5000 g) of 20 g broth in pre-dried (70° C. overnight) and weighted falcons. The pellets were subsequently washed once with 20 ml physiological solution (9 g/l NaCl) and dried at 70° C. to a constant weight. The final weight was corrected for the added sodium chloride to the sample.

Protein Quantification

For protein quantification a method is used that is compatible with reducing agents, such as reducing sugars or oligosaccharides with a reducing end. To this end, a Bradford assay (Thermo Scientific, Pierce) was used with a linear range between 1 and 1500 μg/ml. The assay was calibrated with a standard curve of BSA. The protein content of dried oligosaccharide products was quantified by dissolving a pre-weighed quantify in 18.2 MΩ·cm (Millipore, Bedford, MA, USA) de-ionized water (DIW) up to a quantity of 50% (m/v). The amount of protein is measured at 595 nm and converted to concentration with the calibration curve based on BSA.

DNA Quantification

Production host specific DNA residue is quantified by RT-qPCR, for which specific primers on the host are designed so that residual DNA of the production host in amplified. The RT-qPCR was performed according to the standard protocol of a kit obtained from Sigma and was based on SYBR Green detection.

Total DNA is measured by means of a Threshold assay (Molecular Devices), based on an immunoassay allowing to measure as low as 2 μg of DNA in a sample in solution. Double stranded DNA is measured by means of SPECTRAMAX® QUANT™ ACCUBLUE™ Pico dsDNA Assay Kit (Molecular Devices) having a linear range between 5 μg and 3 ng of dsDNA.

Endotoxin Measurement

Endotoxin in the liquid was measured by means of a LAL test.

Laser Diffraction

The powder particle size can be assessed by laser diffraction. The system detects scattered and diffracted light by an array of concentrically arranged sensor elements. The software-algorithm is then approximating the particle counts by calculating the z-values of the light intensity values, which arrive at the different sensor elements. The analysis can be executed using a SALD-7500 Aggregate Sizer (Shimadzu Corporation, Kyoto, Japan) quantitative laser diffraction system (qLD).

A small amount (spatula tip) of each sample can be dispersed in 2 ml isooctane and homogenized by ultrasonication for five minutes. The dispersion will then be transferred into a batch cell filled with isooctane and analyzed in manual mode.

Data acquisition settings can be as follows: Signal Averaging Count per Measurement: 128, Signal Accumulation Count: 3, and Interval: 2 seconds.

Prior to measurement, the system can be blanked with isooctane. Each sample dispersion will be measured 3 times and the mean values and the standard deviation will be reported. Data can be evaluated using software WING SALD II version V3.1. When the refractive index of the sample is unknown, the refractive index of sugar (disaccharide) particles (1.530) can be used for determination of size distribution profiles. Size values for mean and median diameter are reported. The mean particle sizes for all samples are very similar due to the spray dryer settings used. In addition, the particle size distribution will show the presence of one main size population for all of the samples.

### Example 2. Production of an Oligosaccharide Mixture Comprising 2′FL, 3-FL and DiFL with a Modified E. coli Host in Fed-Batch Fermentations

An E. coli K₁₂ strain modified for GDP-fucose production, as described in the art or as described in Example 1, was sequentially transformed with a first plasmid expressing a constitutive transcriptional unit for the H. pylori alpha-1,2-fucosyltransferase and a second compatible plasmid expressing a constitutive transcriptional unit for the H. pylori alpha-1,3-fucosyltransferase. This modified mutant strain was evaluated in a batch and in a fed-batch fermentation process. Fed-batch fermentations at bioreactor scale (5 and 30 L) were performed as described in Example 1. In these examples, sucrose was used as a carbon source and lactose was added in the batch medium as a precursor. Regular broth samples were taken and the production of 2′FL, 3-FL and DiFL was measured using UPLC as described in Example 1. The experiment demonstrated that broth samples taken at the end of batch phase comprised an oligosaccharide mixture of 2′FL and 3-FL together with unmodified lactose, whereas broth samples taken at the end of the fed-batch phase comprised an oligosaccharide mixture of 2′FL, 3-FL and DiFL. As the ratios of lactose, 2′FL, 3-FL and DiFL changed over time during fed-batch, they could be manipulated during the fermentation process by discontinuation of the fermentation process at a desired time in fed-batch phase.

The resulting broth is clarified as described in Example 12. In a separate example the cells were lysed to increase the release of oligosaccharides as described in Example 11 and further clarified as described in Example 12.

### Example 3. Production of an Oligosaccharide Mixture Comprising LN3, Sialylated LN3, LNT, LSTa and 3′SL in Fermentation Broth of Mutant E. coli Strains when Evaluated in a Fed-Batch Fermentation Process with Glycerol as Carbon Source, Sialic Acid and Lactose as Precursors

An E. coli strain modified to produce LNT, as described in the art or as described in Example 1, was further modified with a genomic knock-out of the E. coli lacZ gene and transformed with an expression plasmid containing constitutive expression cassettes for the NeuA gene from P. multocida coding for N-acylneuraminate cytidylyltransferase and the α-2,3-sialyltransferase gene from P. multocida. This strain produces a mixture of oligosaccharides comprising LN3, 3′-sialylated LN3 (Neu5Ac-a2,3-GlcNAc-b1,3-Gal-b1,4-Glc), LNT, 3′SL and LSTa (Neu5Ac-a2,3-Gal-b1,3-GlcNAc-b1,3-Gal-b1,4-Glc) and was grown in a batch and in a fed-batch fermentation process in a 5 L and 30 L bioreactor. Fed-batch fermentations at bioreactor scale were performed as described in Example 1. In these examples, glycerol was used as a carbon source and lactose was added in the batch medium as precursor. During fed-batch, also sialic acid was added via an additional feed. Regular broth samples were taken, and sugars produced were measured as described in Example 1. UPLC analysis shows that fermentation broth of the selected strain taken after the batch phase contains lactose, LN3 and LNT, whereas fermentation broth of the selected strain taken after the fed-batch phase comprises an oligosaccharide mixture comprising LN3, 3′-sialylated LN3 (Neu5Ac-a2,3-GlcNAc-b1,3-Gal-b1,4-Glc), LNT, LSTa and 3′SL.

The resulting broth is clarified as described in Example 12. In a separate example the cells were lysed to increase the release of oligosaccharides as described in Example 11 and further clarified as described in Example 12.

### Example 4. Production of an Oligosaccharide Mixture Comprising LN3, Sialylated LN3, LNT, LSTa and 3′SL in Fermentation Broth of Mutant E. coli Strains when Evaluated in a Fed-Batch Fermentation Process with Sucrose and Lactose

An E. coli strain modified to produce sialic acid as described in WO 2018122225 was further modified with a genomic knock-in of constitutive transcriptional units for the galactoside beta-1,3-N-acetylglucosaminyltransferase gene (LgtA) from N. meningitidis and for the N-acetylglucosamine beta-1,3-galactosyltransferase gene (WbgO) from E. coli 055: to allow production of LNT. In a next step, the novel strain was further modified with a genomic knock-out of the E. coli lacZ gene and transformed with an expression plasmid having constitutive transcriptional units for the NeuA gene from P. multocida coding for N-acylneuraminate cytidylyltransferase and the α-2,3-sialyltransferase gene from P. multocida. The novel strain produces an oligosaccharide mixture comprising LN3, 3′-sialylated LN3 (Neu5Ac-a2,3-GlcNAc-b1,3-Gal-b1,4-Glc), LNT, 3′SL and LSTa when evaluated in a growth experiment according to the culture conditions provided in Example 1, in which the culture medium contains sucrose as carbon source and lactose as precursor.

This mutant was selected for further evaluation in a fed-batch fermentation process in a 5 L and 30 L bioreactor. Fed-batch fermentations at bioreactor scale were performed as described in Example 1. In these examples, sucrose was used as a carbon source and lactose was added in the batch medium as precursor. Regular broth samples are taken, and sugars produced are measured as described in Example 1. UPLC analysis shows that fermentation broth of the selected strain taken after the batch phase contains lactose, LN3, 3′SL, and LNT, whereas fermentation broth of the selected strain taken after the fed-batch phase comprises an oligosaccharide mixture comprising LN3, 3′-sialylated LN3 (Neu5Ac-a2,3-GlcNAc-b1,3-Gal-b1,4-Glc), LNT, LSTa and 3′SL.

The resulting broth is clarified as described in Example 12. In a separate example the cells were lysed to increase the release of oligosaccharides as described in Example 11 and further clarified as described in Example 12.

### Example 5. Production of an Oligosaccharide Mixture Comprising LN3, Sialylated LN3, LNnT, Para-Lacto-N-Neohexaose, Di-Sialylated LNnT, LSTc and 6′SL in Fermentation Broth of Mutant E. coli Strains when Evaluated in a Fed-Batch Fermentation Process with Glycerol, Sialic Acid and Lactose

An E. coli strain modified to produce LNnT, as described in the art or as described in Example 1, was further modified with a genomic knock-out of the E. coli lacZ gene and transformed with an expression plasmid containing constitutive expression cassettes for the NeuA gene from P. multocida coding for N-acylneuraminate cytidylyltransferase and one selected α-2,6-sialyltransferase gene from P. damselae The strains produce a mixture of oligosaccharides comprising 6′SL, LN3, 6′-sialylated LN3 (Neu5Ac-a2,6-(GlcNAc-b1,3)-Gal-b1,4-Glc), LNnT and LSTc (Neu5Ac-a2,6-Gal-b1,4-GlcNAc-b1,3-Gal-b1,4-Glc).

This mutant strain was cultivated in a batch and fed-batch fermentation process in a 5 L and 30 L bioreactor. Fed-batch fermentations at bioreactor scale were performed as described in Example 1. In these examples, glycerol is used as a carbon source and lactose was added in the batch medium as precursor. During fed-batch, also sialic acid was added via an additional feed. Regular broth samples were taken, and sugars produced were measured as described in Example 1. UPLC analysis shows that fermentation broth of the selected strain taken after the batch phase contains lactose, LN3, and LNnT, whereas fermentation broth of the selected strain taken after the fed-batch phase comprises an oligosaccharide mixture comprising LN3, 6′-sialylated LN3 (Neu5Ac-a2,6-(GlcNAc-b1,3)-Gal-b1,4-Glc), LNnT, LSTc and 6′SL. At end of fed-batch, the mixture also comprises para-lacto-N-neohexaose (pLNnH) and di-sialylated LNnT, two structures that were not detected in growth experiment assays due to limited detection levels and overall smaller production levels.

The resulting broth is clarified as described in Example 12. In a separate example the cells were lysed to increase the release of oligosaccharides as described in Example 11 and further clarified as described in Example 12.

### Example 6. Production of an Oligosaccharide Mixture Comprising LN3, Sialylated LN3, LNnT, Para-Lacto-N-Neohexaose, Di-Sialylated LNnT, LSTc and 6′SL in Fermentation Broth of Mutant E. coli Strains when Evaluated in a Fed-Batch Fermentation Process with Sucrose and Lactose

An E. coli strain modified to produce sialic acid as described in WO 2018122225 was further modified with a genomic knock-in of constitutive transcriptional units for the LgtA gene from N. meningitidis and for the LgtB gene from N. meningitidis to allow production of LNnT. In a next step, the novel strain was further modified with a genomic knock-out of the E. coli lacZ gene and transformed with an expression plasmid having constitutive transcriptional units for the NeuA gene from P. multocida coding for N-acylneuraminate cytidylyltransferase and the α-2,6-sialyltransferase gene from Photobacterium sp. JT-ISH-224. This strain produces a mixture of oligosaccharides comprising LN3, 6′-sialylated LN3 (Neu5Ac-a2,6-(GlcNAc-b1,3)-Gal-b1,4-Glc), 6′SL, LNnT and LSTc.

This strain was grown in a batch and fed-batch fermentation process in a 5 L and 30 L bioreactor. Fed-batch fermentations at bioreactor scale were performed as described in Example 1. In these examples, sucrose was used as a carbon source and lactose was added in the batch medium as precursor. Regular broth samples were taken, and sugars produced were measured as described in Example 1. UPLC analysis shows that fermentation broth of the selected strain taken after the batch phase contains lactose, LN3, 6′SL, and LNnT, whereas fermentation broth of the selected strain taken after the fed-batch phase comprises an oligosaccharide mixture comprising LN3, 6′-sialylated LN3 (Neu5Ac-α-2,6-(GlcNAc-b-1,3)-Gal-b-1,4-Glc), LNnT, LSTc and 6′SL. At end of fed-batch, the mixture also comprises para-lacto-N-neohexaose and di-sialylated LNnT, two structures that were not detected in growth experiment assays due to limited detection levels and overall smaller production levels.

The resulting broth is clarified as described in Example 12. In a separate example the cells were lysed to increase the release of oligosaccharides as described in Example 11 and further clarified as described in Example 12.

### Example 7. Production of an Oligosaccharide Mixture Comprising LNT, LNnT and Poly-Galactosylated Structures in a Modified E. coli Host when Evaluated in Fed-Batch Fermentations

The mutant strain for LNnT, as described in the art or as described in Example 1, is modified with constitutive transcriptional unit of N-acetylglucosamine beta-1,4-galactosyltransferase gene (LgtB) from N. meningitidis in one or more copies. To enhance UDP-galactose production the genes ushA and galT are knocked out. The mutant E. coli strain is further modified with a genomic knock-in of a constitutive transcriptional unit for the UDP-glucose-4-epimerase gene (galE) gene from E. coli, the phosphoglucosamine mutase (glmM) gene from E. coli and the N-acetylglucosamine-1-phosphate uridyltransferase/glucosamine-1-phosphate acetyltransferase (glmU) gene from E. coli. The mutant strain is further mutated for growth on sucrose via genomic knock-ins of constitutive transcriptional units containing a sucrose transporter (CscB) gene from E. coli W, a fructose kinase gene (Frk) originating and a sucrose phosphorylase originating from B. adolescentis. This strain is further modified with genomic knock-ins of constitutive transcriptional units for the WbgO gene from E. coli 055:H7.

The final mutant strain produces a mixture of Lacto-N-triose II (LN3), Lacto-N-neotetraose (LNnT), Lacto-N-tetraose (LNT), para-Lacto-N-neopentaose, para-Lacto-N-pentaose, para-Lacto-N-neohexaose, para-Lacto-N-hexaose, beta-(1,3)Galactosyl-para-Lacto-N-neopentaose and beta-(1,4)Galactosyl-para-Lacto-N-pentaose.

This mutant strain is evaluated in a batch and fed-batch fermentation process in a 5 L and 30 L bioreactor as described in Example 1. In this example sucrose is used as a carbon source and lactose is added in the batch medium as precursor. Regular broth samples are taken, and sugars produced are measured as described in Example 1. UPLC analysis shows that fermentation broth of the selected strain taken at regular timepoints in fed-batch phase contains an oligosaccharide mixture comprising Lacto-N-triose II (LN3), Lacto-N-neotetraose (LNnT), Lacto-N-tetraose (LNT), para-Lacto-N-neopentaose, para-Lacto-N-pentaose, para-Lacto-N-neohexaose, para-Lacto-N-hexaose, beta-(1,3)Galactosyl-para-Lacto-N-neopentaose and beta-(1,4)Galactosyl-para-Lacto-N-pentaose.

The resulting broth is clarified as described in Example 12. In a separate example the cells were lysed to increase the release of oligosaccharides as described in Example 11 and further clarified as described in Example 12.

### Example 8. Production of an Oligosaccharide Mixture Comprising 2′FL, 3-FL, DiFL, LN3, LNT and LNFP-I with a Modified E. coli Host

An E. coli strain modified for GDP-fucose as described in the examples above was further modified to express the glmS*54 gene from E. coli, the LgtA gene from N. meningitidis, the WbgO gene from E. coli O55:H7, the α-1,2-fucosyltransferase gene from H. pylori, and the α-1,3-fucosyltransferase gene (HpFucT). The novel strain produces an oligosaccharide mixture comprising 2′FL, 3-FL, DiFL, LN3, LNT and LNFP-I (Fuc-a1,2-Gal-b1,3-GlcNAc-b1,3-Gal-b1,4-Glc) in a growth experiment according to the culture conditions provided in Example 1, in which the culture medium contains sucrose as carbon source and lactose as precursor. This mutant strain is evaluated in a batch and fed-batch fermentation process in a 5 L and 30 L bioreactor as described in Example 1. In this example sucrose is used as a carbon source and lactose is added in the batch medium as precursor. Regular broth samples are taken, and sugars produced are measured as described in Example 1. UPLC analysis shows that fermentation broth of the selected strain taken at regular timepoints in fed-batch phase contains an oligosaccharide mixture comprising 2′FL, 3-FL, DiFL, LN3, LNT and LNFP-I (Fuc-a1,2-Gal-b1,3-GlcNAc-b1,3-Gal-b1,4-Glc).

The resulting broth is clarified as described in Example 12. In a separate example the cells were lysed to increase the release of oligosaccharides as described in Example 11 and further clarified as described in Example 12.

### Example 9. Production of an Oligosaccharide Mixture Comprising Fucosylated and Sialylated Oligosaccharide Structures with a Modified E. coli Host

An E. coli strain adapted for sialic acid production as described in WO 2018122225 was further modified with a genomic knock-out of the E. coli wcaJ gene to increase the intracellular pool of GDP-fucose and genomic knock-ins of constitutive expression cassettes for the LgtA gene from N. meningitidis and the WbgO gene from E. coli O55:H7. In a next step, the novel strain was transformed with two compatible expression plasmids wherein a first plasmid pMF_2 contained (a) constitutive expression unit(s) for two fucosyltransferase genes, H. pylori alpha-1,2-fucosyltransferase gene (HpFutC) and the H. pylori alpha-1,3-fucosyltransferase gene (HpFucT), and wherein a second plasmid pMS_2 contained constitutive expression units for two sialyltransferase genes, alpha-2,3-sialyltransferase from P. multocida and alpha-2,6-sialyltransferase (PdST6) from Photobacterium damselae, and the NeuA gene from P. multocida coding for N-acylneuraminate cytidylyltransferase. This strain produces an oligosaccharide mixture comprising fucosylated and sialylated lactose, LNB, fucosylated and sialylated LNB, LN3, sialylated LN3, LNT and fucosylated and sialylated LNT structures in whole broth samples. The strain was grown in an experiment according to the culture conditions provided in Example 1 in which the cultivation contains sucrose as carbon source and lactose as precursor.

This mutant strain is evaluated in a batch and fed-batch fermentation process in a 5 L and 30 L bioreactor as described in Example 1. In this example sucrose is used as a carbon source and lactose is added in the batch medium as precursor. Regular broth samples are taken, and sugars produced are measured as described in Example 1. UPLC analysis shows that fermentation broth of the selected strain taken at regular timepoints in fed-batch phase contains an oligosaccharide mixture comprising 2′FL, 3-FL, DiFL, 3′SL, 6′SL, di-SL, 3'S-2′FL, 3'S-3-FL, 6'S-2′FL, 6'S-3-FL, LNB, 2′FLNB, 4-FLNB, Di-FLNB, 3′SLNB, 6′SLNB, LN3, 3'S-LN3, 6'S-LN3, LNT, LNFP-I, LSTa.

The resulting broth is clarified as described in Example 12. In a separate example the cells were lysed to increase the release of oligosaccharides as described in Example 11 and further clarified as described in Example 12.

### Example 10. Composition Determination of the Fermentation Broth

For the fermentation broths obtained in Examples 2-9 the composition was determined by measuring the Cell dry mass of the broth, the ash content of the supernatant and the broth, the oligosaccharide content of the supernatant and the broth and the total dry solids in the broth in accordance to the methods described in Example 1. For all samples the total oligosaccharide content was below 80% on total dry solids. The oligosaccharide mixture purity in the broth ranged from 30% to 77%.

### Example 11. Cell Lysis

In many of the above described mutant strains the product is readily excreted from the cell. Larger molecules however tend to be released more difficult during the fermentation process. Therefore an additional step is optionally introduced to release the product from the cell. The broth from the fermentation processes of Examples 2-8 is used in a cell lysis experiment.

A soft release of the product was established by heating for 1 h the broth to a temperature between 60° C. and 80° C. The higher the temperature, the more release was obtained, but color formation increased. The product release was most optimal at a pH below 6.5 and above 3. The least monosaccharide formation was found at a pH of above 3.9. The release of the product is quantified by the measured of the total oligosaccharide pool (cfr methods Example 1) in the broth before and after treatment. When observing an increase in oligosaccharide concentration, the product is released from the cells.

To disrupt the cell integrity even more other methods are also commonly used, these are methods such as, freeze thawing and/or shear stress through sonication, mixing, a homogenizer and/or French press.

### Example 12. Broth Clarification

The broth originating from the cultivation or fermentation and, as the case may be, lysis step described in Examples 2-11 are further clarified through microfiltration. For filtration several types of microfiltration membranes have been used to clarify the fermentation broth with a pore size ranging between 0.1 to 10 m (ceramic, PES, PVDF membranes). The membrane types were first used as dead-end filtration and further optimization was performed in cross flow filtration. The cross-flow microfiltration was followed by diafiltration to increase product yield after this purification step. The membranes are capable of separating large suspended solids such as colloids, particulates, fat, bacteria, yeasts, fungi, cells, while allowing sugars, proteins, salts, and low molecular weight molecules pass through the membrane.

The particle concentration in the filtrate was measured with a spectrophotometer through at light adsorption at 600 nm. This method allows the validation of particle removal and filtration optimization.

Alternative to microfiltration membranes, ultrafiltration membranes are used. Ultrafiltration membranes with a cut-off between 1000 Da and 10 kDa were tested (microdyne Nadir (3 kDa PES), Synder (3 kDa, PES), Synder Filtration MT (5 kDa, PES) and Synder Filtration ST (10 kDa, PES)). Alternative membranes with larger cut-offs will also work for broth clarification. The membranes were used in cross flow mode, and diafiltrations were applied similar to the microfiltration operation described above to increase product yield. The filtration efficiency is evaluated based on the particle concentration of the filtrate. Apart from cells and cell debris, membranes below 10 kDa efficiently remove DNA, protein and endotox, which were measured with the methods described in Example 1. Higher cut-off membranes between 10 and 500 kDa remove cell mass efficiently, but do not retain smaller molecular weight products as efficiently, therefore requiring an additional Ultrafiltration step with a molecular weight cut-off below 10 kDa. A final recovery through ultrafiltration for broth clarification of above 95% was obtained.

To enhance broth clarification through centrifugation, flocculants/coagulants have been used. Generally, Gypsum, Alum, calcium hydroxide, polyaluminum chloride, Aluminum chlorohydrate, are used as good flocculation agents. These flocculants were applied at a pH>7 and at temperatures between 4° C. and 20° C., more preferably between 4° C. and 10° C. pH<7 released toxic cations that are removed further through cation exchange. Alternative flocculants tested are based on polyacrylamide or biopolymer (chitosan), Floquant (SNF Inc.), SUPERFLOC® (Kemira) or HYPERFLOC® (Hychem Inc.), TRAMFLOC®. These flocculants were used in different concentrations: 0.05, 0.1 and 0.2 v/v % after diluting the broth 1:1 with RO-water, they were directly added to the broth and gently mixed for 10 minutes at room temperature. pH was kept at neutral conditions, between pH 6 and 7. At higher pH some degradation of the flocculant occurs, leading to compounds that are removed by means of ion exchange.

To test flocculation efficiency centrifugation was performed at 4000 g and the pellet strength and supernatant turbidity was evaluated after different centrifugation times. The oligosaccharide yield was measured by measuring the oligosaccharide supernatant concentration and the total supernatant volume. The pellet was washed several times to increase the release of oligosaccharides. A final oligosaccharide recovery between 90 and 98% was obtained.

### Example 13. Ultrafiltration

Ultrafiltration was performed on a Colossus apparatus (Convergence Industry, The Netherlands) controlled by a PC running Convergence Inspector software. Temperature, pressures and conductivity of both retentate and filtrate were measured inline, pH was measured offline with a calibrated pH probe (Hanna Instruments). The membrane to further remove DNA, protein and endotoxin was a 10 kDa membrane based on PES (Synder), used in crossflow. After filtration, the DNA, protein and endotoxin content was measured in the filtrate. The protein content was below 100 mg per kg dry solid, the DNA content below 10 ng per gram dry solid and the endotoxin was below 10000 EU per gram dry solid. No DNA from the production hosts could be detected in the filtrate.

Although in this example a polysulfon based membrane was used, other membrane materials will perform equally, these membrane materials can be a ceramic or made of a synthetic or natural polymer, e.g., polypropylene, cellulose acetate or polylactic acid from suppliers such as Synder, Tami, TriSep, Microdyn Nadir, GE.

### Example 14. Ion and Disaccharide Removal Through Nanofiltration

Tangential flow nanofiltration was performed on a Colossus apparatus (Convergence Industry, The Netherlands) controlled by a PC running Convergence Inspector software. Temperature, pressures and conductivity of both retentate and filtrate were measured inline, pH was measured offline with a calibrated pH probe (Hanna Instruments). Clarified liquid treated with ultrafiltration from Example 13 was further subjected to nanofiltration and sequential diafiltrations. To this end a polyamide base membrane with a cut off between 300 and 500 Da was used (TriSep XN-45 (TriSep Corporation, USA) at 40° C. The diafiltrations were done with deionized water with a total volume of 5 times the volume of the oligosaccharide mixture concentrate. This step reduced the disaccharide fraction on dry solid below 5% and reduced the total ash content of the liquid with 50%. The concentration of the oligosaccharide mixture was increased to about 200 g/1.

### Example 15. Ion Removal Through Electrodialysis

The ED equipment used is a PCCell ED 64004 lab-scale ED stack, fitted with 5 cell pairs of the PC SA and PC SK standard ion-exchange membranes. The initial diluate and concentrate both comprised 1.5 L of the feed stream obtained after the clarification and ultrafiltration in Examples 12 and 13. The liquids obtained in these examples contained oligosaccharides and cations and anions with an ash content above 10% on dry solid. The desalination was done against a concentration gradient. Both streams are recirculated while a constant voltage of 7.5V is applied and the current and conductivity are monitored. Samples are taken at the beginning and end and periodically during the experiment. Water transport across the membranes is monitored by measuring the volume of all streams at the end of the experiment. To ensure efficient transfer of the current to the stack, an electrolyte solution of 60 g/L NaNO₃ is recirculated at the electrodes.

The ED experiment was maintained until a stabilization of the current and conductivity was noticed. This indicates the point where desalination becomes slow and more inefficient. The conductivity decreases from 3.79 mS/cm in the feed to 0.88 mS/cm at the end of the experiment, indicating an overall desalination of 77%. The multivalent anions were removed up to 90%. The final oligosaccharide recovery was between 90 and 99%. The ash content on dry solid after electrodialysis was about 2.5% on dry solid.

### Example 16. Ion Removal Through Ion Exchange

To remove ions from the broth to an ash content<1%, first and cation exchange and second an anion exchange step was performed. Depending on the mixture of oligosaccharides different anion exchange resins were selected to enhance the yield of the purification step.

For clarified broths originating from Examples 8, 7 and 2, containing non-charged oligosaccharides were first passed through a strong acid cation exchange resin containing column (1 L of AMBERLITE™ IR120) in the proton form at a temperature of 10° C., resulting in exchange of all cations with a proton in the liquid. The liquid resulting from the cation exchange step was subjected to a weak base anion exchange resin containing column (1 L of AMBERLITE™ IR400) in the hydroxide form at a temperature of 10° C., exchanging the anions in the liquid for hydroxide ions. After both cation and anion exchange, the pH was set to a pH between 6 and 7. The oligosaccharide recovery was between 95 and 98%.

Alternative cation and anion exchange resins are sold under the tradenames AMBERLITE™ IR100, AMBERLITE™ IR120, AMBERLITE™ FPC22, DOWEX™ 50WX, FINEX™ CS16GC, FINEX™ CS13GC, FINEX™ CS12GC, FINEX™ CS11GC, LEWATIT™ S, DIAION™ SK, DIAION™ UBK, AMBERJET™ 1000, AMBERJET™ 1200 and AMBERJET™ 4200, AMBERJET™ 4600, AMBERLITE™ IR400, AMBERLITE™ IR410, AMBERLITE™ IR458, DIAION™ SA, DIAION™ UBA120, LEWATIT MONOPLUS™ M, and LEWATIT™ S7468.

The cation and anion exchange treated liquids were then tested on ash, oligosaccharide content and heavy metal content. The ash content after treatment was below 0.5% (on total dry solid), the Lead content was lower than 0.1 mg/kg dry solid, Arsenic: lower than 0.2 mg/kg dry solid, Cadmium lower than 0.1 mg/kg dry solid and Mercury was lower than 0.5 mg/kg dry solid.

For clarified broths originating from Examples 3, 4, 5, 6, and 9 specific anion exchange resins were used that do not retain the charged oligosaccharides (containing a sialyl group). These resins are characterized to have a moisture content of 30-48% and preferably a gel type anion exchanger. Examples of such resins are DIAION™ SA20A, DIAION™ WA20A (Mitsubishi), XA4023 (Applexion), DOWEX™ 1-X8 (Dow). In a first step the liquid was first passed through a strong acid cation exchange resin containing column (1 L of AMBERLITE™ IR120) in the proton form at a temperature of 10° C., resulting in exchange of all cations with a proton in the liquid. This was then passed immediately through an anion exchange resin column (1 L of XA4023), exchanging salts like phosphates and sulphates for hydroxide ions. The resulting liquid was set to a pH between 5 and 7. The ash content corrected for the sodium counter ions for the sialylated oligosaccharides was below 1% (on total dry solid) after ion exchange treatment, the Lead content was lower than 0.1 mg/kg dry solid, Arsenic: lower than 0.2 mg/kg dry solid, Cadmium lower than 0.1 mg/kg dry solid and Mercury was lower than 0.5 mg/kg dry solid.

An alternative to sequential cation and anion exchange steps is mixed bed ion exchange. The resins are mixed in a ratio typically within the range of 35:65 and 65:35 volume percentage. Typically, a mixed bed ion exchange step is introduced in the process after a first de-ionization step such as a nanofiltration step, an electrodialysis step or ion exchange step but is also used as sole ion exchange step. For the oligosaccharide mixtures obtained in Examples 8, 7 and 2, the clarified broth after ultrafiltration in Example 13 was subjected to a mixed bed column of AMBERLITE™ FPC 22H and AMBERLITE™ FPA51 mixed in a ratio 1:1.3 on a 1 L column. The mixed bed step was performed at a temperature between 4° C. and 10° C. Finally, the liquid was set to a pH between 5 and 7 and the ash content of the solution was measured to be below 1%. The oligosaccharide recovery was between 95 and 98%.

For clarified broths originating from Examples 3, 4, 5, 6, and 9, after ultrafiltration in Example 13, the liquids were subjected to a mixed bed column of DIAION™ SA20A and AMBERLITE™ FPC 22H mixed in a ratio 1,3:1 on a 1 L column. Similar to the above the mixed bed step was performed at a temperature between 4° C. and 10° C. Finally, the liquid was set to a pH between 5 and 7 and the ash content of the solution was measured to be below 1%. None of the sialylated oligosaccharides were retained in this step, retaining the mixture composition, the oligosaccharide recovery was between 95 and 98%.

### Example 17. Concentration Through Nanofiltration

Nanofiltration was carried out with an NF-2540 membrane (DOW) with a cut off of 200 Da to concentrate the de-ionized solutions after ion exchange, electrodialysis or nanofiltration up to 25 Brix. During the filtration process a pressure across the membrane in the range of 20-25 bar was used and a process temperature of 45° C. The solution was continuous recirculated over the membrane for concentration, leading to a dry matter content of the concentrate up to 25% Brix.

### Example 18. Color Removal

To achieve decolorization, several samples throughout the process were subjected to activated charcoal treatment with NORIT™ SX PLUS activated charcoal (0.5% m/v). Color removal was measured with a spectrophotometer at 420 nm. In all samples the color intensity at 420 nm was reduced 50- to 100-fold. The activated charcoal is filtered of by means of a plate filter or chamber filter press preferably at elevated temperatures.

### Example 19. Spray Drying of Oligosaccharide Mixture

A mixture of oligosaccharides at different concentrations was spray dried with pilot spray dry equipment. The equipment had an evaporation capacity of 25 kg/h.

For spray drying the liquid was heated to a temperature between 50 and 100° C., to lower the viscosity. The pH of the liquid was set to a pH of 4 to 6. More preferably the pH is set to 4 to 5 and temperatures are kept between 50 and 70° C.

The oligosaccharide concentration in the feed is between 20% and 80% brix. These concentrations were obtained by rotary evaporation or wiped film evaporation. The concentrated liquids were fed to the spray dryer at a rate between 50% and 90%. The higher the percentage brix, the faster the feed rate.

The used inlet temperature ranged between 120° C. and 280° C. The outlet temperature ranged between 100° C. and 180° C. The atomizer wheel rotation speed was set between 10000 rpm and 28000 rpm. In one specific test the inlet temperature was set at 184° C., outlet temperature was set at 110° C. and atomizer rate was set at 21500 rpm.

The obtained powder had a white to off white color and the pH after re-dissolving water at a concentration of 10%, the pH was between 4 and 6. The purity of the oligosaccharide mixture was above 80% of oligosaccharides on dry solid. The spray dried oligosaccharide mixtures had about 3% to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 1% (on total dry solid), the Lead content was lower than 0.1 mg/kg dry solid, Arsenic: lower than 0.2 mg/kg dry solid, Cadmium lower than 0.1 mg/kg dry solid and Mercury was lower than 0.5 mg/kg dry solid.

### Example 20. Stepwise Purification of a Fucosylated Oligosaccharide Mixture

The broth of Example 8 was clarified by first applying microfiltration with a 0.45 μm pore sized membrane, removing biomass at 60° C. and a pH of 4 to 5. The filtrate of the microfiltration step was in a second step subjected to ultrafiltration in which a PES membrane of 10 kDa was used, removing protein, endotoxin and DNA. The resulting filtrate was further concentrated by nanofiltration, partially removing salts and disaccharides from the liquid with a polyamide membrane of 300 Da to 500 Da at 40° C. In the nanofiltration step the oligosaccharide mixture was concentrated to a concentration of about 200 g/l or 20 Brix. The resulting concentrate was further decolored by means of activated charcoal and de-ionized with a cation exchange step and an anion exchange step resulting in an ash content below 1% on dry mass. This de-ionized liquid was set to a pH between 5 and 7 and concentrated by means of evaporation to about 50 brix. The final solution was spray dried with an inlet temperature of 160° C., outlet temperature of 75° C., an airflow of 600 L/h and a feed rate of 8 ml/min on a Procept spray dryer. The obtained powder had a white to off white color and the pH after re-dissolving water at a concentration of 10%, the pH was between 4 and 6. The purity of the oligosaccharide mixture was above 80% of oligosaccharides on dry solid. The spray dried oligosaccharide mixtures had about 3 to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 1% (on total dry solid), the Lead content was lower than 0.1 mg/kg dry solid, Arsenic: lower than 0.2 mg/kg dry solid, Cadmium lower than 0.1 mg/kg dry solid and Mercury was lower than 0.5 mg/kg dry solid. The oligosaccharides present in the obtained powder are 2′FL, 3-FL, DiFL, LN3, LNT and LNFP-I.

### Example 21. Stepwise Purification of a Sialylated Oligosaccharide Mixture

The broth of Example 6 was clarified by first applying microfiltration with a 0.45 μm pore sized membrane, removing biomass at 60° C. and a pH of 4 to 5. The filtrate of the microfiltration step was in a second step subjected to ultrafiltration in which a PES membrane of 10 kDa was used, removing protein, endotoxin and DNA. The resulting filtrate was further concentrated and deionized with electrodialysis resulting into a liquid solution with a conductivity of about 0.9 mS/cm. After the electrodialysis step, the retentate is further treated in a nanofiltration step concentrating the oligosaccharide mixture to a concentration of about 200 g/l or 20 Brix. The resulting concentrate was further decolored by means of activated charcoal and de-ionized with a cation exchange step and an anion exchange step resulting in an ash content below 1% on dry mass. This de-ionized liquid was set to a pH between 5 and 7 and concentrated by means of evaporation to about 50 brix. The final solution was spray dried with an inlet temperature of 160° C., outlet temperature of 75° C., an airflow of 600 L/h and a feed-rate of 8 ml/min on a Procept spray dryer. The obtained powder had a white to off white color and the pH after redissolving water at a concentration of 10%, the pH was between 4 and 7. The purity of the oligosaccharide mixture was above 80% of oligosaccharides on dry solid. The spray dried oligosaccharide mixtures had about 3% to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 1% (on total dry solid), the Lead content was lower than 0.1 mg/kg dry solid, Arsenic: lower than 0.2 mg/kg dry solid, Cadmium lower than 0.1 mg/kg dry solid and Mercury was lower than 0.5 mg/kg dry solid. The oligosaccharides present in the obtained powder are LN3, 6′-sialylated LN3 (Neu5Ac-α-2,6-(GlcNAc-b-1,3)-Gal-b-1,4-Glc), LNnT, LSTc, and 6′SL.

### Example 22. Stepwise Purification of a Sialylated and Fucosylated Oligosaccharide Mixture

The broth of Example 9 was clarified by first applying microfiltration with a 0.45 μm pore sized membrane, removing biomass at 60° C. and a pH of 4 to 5. The filtrate of the microfiltration step was in a second step subjected to ultrafiltration in which a PES membrane of 10 kDa was used, removing protein, endotoxin and DNA. The resulting filtrate was further concentrated by nanofiltration, partially removing salts and disaccharides from the liquid with a polyamide membrane of 300 Da to 500 Da at 40° C. In the nanofiltration step the oligosaccharide mixture was concentrated to a concentration of about 200 g/l or 20 Brix. The resulting concentrate was further decolored by means of activated charcoal and de-ionized with a cation exchange step and an anion exchange step resulting in an ash content below 1% on dry mass. This de-ionized liquid was set to a pH between 5 and 7 and concentrated by means of evaporation to about 50 brix. The final solution heated to 70° C. and was spray dried with an inlet temperature of 184° C., outlet temperature of 104° C., atomizer speed 21800 rpm, at a feed rate of 66% on an Anhydro spray dryer. The obtained powder had a white to off white color and the pH after redissolving water at a concentration of 10%, the pH was between 4 and 7. The purity of the oligosaccharide mixture was above 80% of oligosaccharides on dry solid. The spray dried oligosaccharide mixtures had about 3% to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 1% (on total dry solid), the Lead content was lower than 0.1 mg/kg dry solid, Arsenic: lower than 0.2 mg/kg dry solid, Cadmium lower than 0.1 mg/kg dry solid and Mercury was lower than 0.5 mg/kg dry solid. The oligosaccharides present in the obtained powder are 2′FL, 3-FL, DiFL, 3′SL, 6′SL, di-SL, 3'S-2′FL, 3'S-3-FL, 6'S-2′FL, 6'S-3-FL, LNB, 2′FLNB, 4-FLNB, Di-FLNB, 3′SLNB, 6′SLNB, LN3, 3'S-LN3, 6'S-LN3, LNT, LNFP-I, and LSTa.

### Example 23. Purification of a Mixture of Sulphates, Deacetylated and Non-Deacetylated Chitosan Oligosaccharides

The production of a mixture in a fermentation process was performed in accordance to Example 1 and based on previous work by Samain et al (1999) (E. Samain et al. Journal of Biotechnology 72 (1999) 33-47). At the end of the fermentation the fermentation broth pH was reduced to a pH of 5 and was heated to a temperature of 70° C. for 1 hour to release the oligosaccharide mixture. The broth with the lysed cells was further clarified by means of a microfiltration step with a 0.22 μm ceramic filter. The filtrate was then treated over a 3 kDa ceramic membrane to remove endotoxin, protein and DNA. Thereafter the oligosaccharide mixture was concentrated to 10 Brix and ions were removed by means of electrodialysis to an ash content below 1%. After electrodialysis the pH was set between 5 and 7. Retentate was further treated by means of a cation exchange resin and anion exchange resin at room temperature and the eluate was again set to a pH of 5 to 7. This was then concentrated in a rotary evaporator and treated with activated charcoal to decolor. The charcoal was filtered off by means of plate filtration and the final filtrate was spray dried on a Procept spray dryer.

### Example 24. Examples of Stepwise Implementations of this Disclosure

Each of the steps described hereunder must be understood as described herein.

Purification of an oligosaccharide mixture obtained from a cultivation or fermentation process, comprising the following steps 1) broth clarification, 2) ultrafiltration, 3) nanofiltration, 4) cation exchange, 5) anion exchange, 6) concentration, and 7) spray drying.

Purification of an oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, and 7) spray drying.

Purification of an oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, and 7) spray drying.

Purification of an oligosaccharide mixture obtained from a cultivation or fermentation process, comprising the following steps 1) broth clarification through ultrafiltration, 2) nanofiltration, 3) cation exchange, 4) anion exchange, 5) concentration, and 6) spray drying.

Purification of an oligosaccharide mixture obtained from a cultivation or fermentation process, comprising the following steps 1) broth clarification, 2) ultrafiltration, 3) electrodialysis, 4) cation exchange, 5) anion exchange, 6) concentration, and 7) spray drying.

Purification of an oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, and 7) spray drying.

Purification of an oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, and 7) spray drying.

Purification of an oligosaccharide mixture obtained from a cultivation or fermentation process, comprising the following steps 1) broth clarification through ultrafiltration, 2) electrodialysis, 3) cation exchange, 4) anion exchange, 5) concentration, and 6) spray drying.

Purification of an oligosaccharide mixture obtained from a cultivation or fermentation process, comprising the following steps 1) broth clarification, 2) ultrafiltration, 3) nanofiltration, 4) mixed bed ion exchange, 5) concentration, and 6) spray drying.

Purification of an oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, and 6) spray drying.

Purification of an oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, and 6) spray drying.

Purification of an oligosaccharide mixture obtained from a cultivation or fermentation process, comprising the following steps 1) broth clarification through ultrafiltration, 2) nanofiltration, 4) mixed bed ion exchange, 5) concentration, and 6) spray drying.

Purification of an oligosaccharide mixture obtained from a cultivation or fermentation process, comprising the following steps 1) broth clarification, 2) ultrafiltration, 3) electrodialysis, 4) mixed bed ion exchange, 5) concentration, and 6) spray drying.

Purification of an oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, and 6) spray drying.

Purification of an oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, and 6) spray drying.

Purification of an oligosaccharide mixture obtained from a cultivation or fermentation process, comprising the following steps 1) broth clarification through ultrafiltration, 2) electrodialysis, 4) mixed bed ion exchange, 5) concentration, and 6) spray drying.

Purification of an oligosaccharide mixture obtained from a cultivation or fermentation process, comprising the following steps 1) broth clarification, 2) ultrafiltration, 3) nanofiltration, 4) cation exchange, 5) anion exchange, 6) concentration, 7) Activated Charcoal treatment, and 8) spray drying.

Purification of an oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, 7) Activated Charcoal treatment, and 8) spray drying.

Purification of an oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, 7) Activated Charcoal treatment, and 8) spray drying.

Purification of an oligosaccharide mixture obtained from a cultivation or fermentation process, comprising the following steps 1) broth clarification through ultrafiltration, 2) nanofiltration, 3) cation exchange, 4) anion exchange, 5) concentration, 6) Activated Charcoal treatment, and 7) spray drying.

Purification of an oligosaccharide mixture obtained from a cultivation or fermentation process, comprising the following steps 1) broth clarification, 2) ultrafiltration, 3) electrodialysis, 4) cation exchange, 5) anion exchange, 6) concentration, 7) Activated Charcoal treatment, and 8) spray drying.

Purification of an oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, 7) Activated Charcoal treatment, and 8) spray drying.

Purification of an oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, 7) Activated Charcoal treatment, and 8) spray drying.

Purification of an oligosaccharide mixture obtained from a cultivation or fermentation process, comprising the following steps 1) broth clarification through ultrafiltration, 2) electrodialysis, 3) cation exchange, 4) anion exchange, 5) concentration, 6) Activated Charcoal treatment, and 7) spray drying.

Purification of an oligosaccharide mixture obtained from a cultivation or fermentation process, comprising the following steps 1) broth clarification, 2) ultrafiltration, 3) nanofiltration, 4) mixed bed ion exchange, 5) concentration, 6) Activated Charcoal treatment, and 7) spray drying.

Purification of an oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, 6) Activated Charcoal treatment, and 7) spray drying.

Purification of an oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, 6) Activated Charcoal treatment, and 7) spray drying.

Purification of an oligosaccharide mixture obtained from a cultivation or fermentation process, comprising the following steps 1) broth clarification through ultrafiltration, 2) nanofiltration, 4) mixed bed ion exchange, 5) concentration, 6) Activated Charcoal treatment, and 7) spray drying.

Purification of an oligosaccharide mixture obtained from a cultivation or fermentation process, comprising the following steps 1) broth clarification, 2) ultrafiltration, 3) electrodialysis, 4) mixed bed ion exchange, 5) concentration, 6) Activated Charcoal treatment, and 7) spray drying.

Purification of an oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, 6) Activated Charcoal treatment, and 7) spray drying.

Purification of an oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, 6) Activated Charcoal treatment, and 7) spray drying.

Purification of an oligosaccharide mixture obtained from a cultivation or fermentation process, comprising the following steps 1) broth clarification through ultrafiltration, 2) electrodialysis, 4) mixed bed ion exchange, 5) concentration, 6) Activated Charcoal treatment, and 7) spray drying.

Purification of an oligosaccharide mixture obtained from a cultivation or fermentation process comprising the following steps 1) broth clarification, 2) ultrafiltration, 3) cation exchange, 4) anion exchange, 5) concentration, 6) Activated Charcoal treatment, and 7) spray drying.

Purification of an oligosaccharide mixture obtained from a cultivation or fermentation process, comprising the following steps 1) broth clarification, 2) ultrafiltration, 3) nanofiltration, 4) cation exchange, 5) anion exchange, 6) concentration, and 7) lyophilization.

Purification of an oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, and 7) lyophilization.

Purification of an oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, and 7) lyophilization.

Purification of an oligosaccharide mixture obtained from a cultivation or fermentation process, comprising the following steps 1) broth clarification through ultrafiltration, 2) nanofiltration, 3) cation exchange, 4) anion exchange, 5) concentration, and 6) lyophilization.

Purification of an oligosaccharide mixture obtained from a cultivation or fermentation process, comprising the following steps 1) broth clarification, 2) ultrafiltration, 3) electrodialysis, 4) cation exchange, 5) anion exchange, 6) concentration, and 7) lyophilization.

Purification of an oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, and 7) lyophilization.

Purification of an oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, and 7) lyophilization.

Purification of an oligosaccharide mixture obtained from a cultivation or fermentation process, comprising the following steps 1) broth clarification through ultrafiltration, 2) electrodialysis, 3) cation exchange, 4) anion exchange, 5) concentration, and 6) lyophilization.

Purification of an oligosaccharide mixture obtained from a cultivation or fermentation process, comprising the following steps 1) broth clarification, 2) ultrafiltration, 3) nanofiltration, 4) mixed bed ion exchange, 5) concentration, 6) lyophilization.

Purification of an oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, and 6) lyophilization.

Purification of an oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, and 6) lyophilization.

Purification of an oligosaccharide mixture obtained from a cultivation or fermentation process, comprising the following steps 1) broth clarification through ultrafiltration, 2) nanofiltration, 4) mixed bed ion exchange, 5) concentration, and 6) lyophilization.

Purification of an oligosaccharide mixture obtained from a cultivation or fermentation process, comprising the following steps 1) broth clarification, 2) ultrafiltration, 3) electrodialysis, 4) mixed bed ion exchange, 5) concentration, and 6) lyophilization.

Purification of an oligosaccharide mixture obtained from a cultivation or fermentation process, comprising the following steps 1) broth clarification through microfiltration, 2) ultrafiltration, 3) electrodialysis, 4) mixed bed ion exchange, and 5) concentration, 6) lyophilization.

Purification of an oligosaccharide mixture obtained from a cultivation or fermentation process, comprising the following steps 1) broth clarification through centrifugation, 2) ultrafiltration, 3) electrodialysis, 4) mixed bed ion exchange, and 5) concentration, 6) lyophilization.

Purification of an oligosaccharide mixture obtained from a cultivation or fermentation process, comprising the following steps 1) broth clarification through ultrafiltration, 2) electrodialysis, 4) mixed bed ion exchange, 5) concentration, and 6) lyophilization.

Purification of an oligosaccharide mixture obtained from a cultivation or fermentation process, comprising the following steps 1) broth clarification, 2) ultrafiltration, 3) nanofiltration, 4) cation exchange, 5) anion exchange, 6) concentration, 7) Activated Charcoal treatment and 8) lyophilization.

Purification of an oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, 7) Activated Charcoal treatment, and 8) lyophilization.

Purification of an oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, 7) Activated Charcoal treatment, and 8) lyophilization.

Purification of an oligosaccharide mixture obtained from a cultivation or fermentation process, comprising the following steps 1) broth clarification through ultrafiltration, 2) nanofiltration, 3) cation exchange, 4) anion exchange, 5) concentration, 6) Activated Charcoal treatment, and 7) lyophilization.

Purification of an oligosaccharide mixture obtained from a cultivation or fermentation process, comprising the following steps 1) broth clarification, 2) ultrafiltration, 3) electrodialysis, 4) cation exchange, 5) anion exchange, 6) concentration, 7) Activated Charcoal treatment, and 8) lyophilization.

Purification of an oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, 7) Activated Charcoal treatment, and 8) lyophilization.

Purification of an oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, 7) Activated Charcoal treatment, and 8) lyophilization.

Purification of an oligosaccharide mixture obtained from a cultivation or fermentation process, comprising the following steps 1) broth clarification through ultrafiltration, 2) electrodialysis, 3) cation exchange, 4) anion exchange, 5) concentration, 6) Activated Charcoal treatment, and 7) lyophilization.

Purification of an oligosaccharide mixture obtained from a cultivation or fermentation process, comprising the following steps 1) broth clarification, 2) ultrafiltration, 3) nanofiltration, 4) mixed bed ion exchange, 5) concentration, 6) Activated Charcoal treatment 7) lyophilization.

Purification of an oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, 6) Activated Charcoal treatment, and 7) lyophilization.

Purification of an oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, 6) Activated Charcoal treatment, and 7) lyophilization.

Purification of an oligosaccharide mixture obtained from a cultivation or fermentation process, comprising the following steps 1) broth clarification through ultrafiltration, 2) nanofiltration, 4) mixed bed ion exchange, 5) concentration, 6) Activated Charcoal treatment, and 7) lyophilization.

Purification of an oligosaccharide mixture obtained from a cultivation or fermentation process, comprising the following steps 1) broth clarification, 2) ultrafiltration, 3) electrodialysis, 4) mixed bed ion exchange, 5) concentration, 6) Activated Charcoal treatment, and 7) lyophilization.

Purification of an oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, 6) Activated Charcoal treatment, and 7) lyophilization.

Purification of an oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, 6) Activated Charcoal treatment, and 7) lyophilization.

Purification of an oligosaccharide mixture obtained from a cultivation or fermentation process, comprising the following steps 1) broth clarification through ultrafiltration, 2) electrodialysis, 4) mixed bed ion exchange, 5) concentration, 6) Activated Charcoal treatment, and 7) lyophilization.

Purification of an oligosaccharide mixture obtained from a cultivation or fermentation process comprising the following steps 1) broth clarification, 2) ultrafiltration, 3) cation exchange, 4) anion exchange, 5) concentration, 6) Activated Charcoal treatment, and 7) lyophilization.

Purification of an oligosaccharide mixture obtained from a cultivation or fermentation process, comprising the following steps 1) broth clarification, 2) ultrafiltration, 3) nanofiltration, 4) cation exchange, 5) anion exchange, 6) concentration, and 7) concentrating to a syrup of at least 40% dry matter.

Purification of an oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, and 7) concentrating to a syrup of at least 40% dry matter.

Purification of an oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, and 7) concentrating to a syrup of at least 40% dry matter.

Purification of an oligosaccharide mixture obtained from a cultivation or fermentation process, comprising the following steps 1) broth clarification through ultrafiltration, 2) nanofiltration, 3) cation exchange, 4) anion exchange, 5) concentration, and 6) concentrating to a syrup of at least 40% dry matter.

Purification of an oligosaccharide mixture obtained from a cultivation or fermentation process, comprising the following steps 1) broth clarification, 2) ultrafiltration, 3) electrodialysis, 4) cation exchange, 5) anion exchange, 6) concentration, and 7) concentrating to a syrup of at least 40% dry matter.

Purification of an oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, and 7) concentrating to a syrup of at least 40% dry matter.

Purification of an oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, 7) concentrating to a syrup of at least 40% dry matter.

Purification of an oligosaccharide mixture obtained from a cultivation or fermentation process, comprising the following steps 1) broth clarification through ultrafiltration, 2) electrodialysis, 3) cation exchange, 4) anion exchange, 5) concentration, 6) concentrating to a syrup of at least 40% dry matter.

Purification of an oligosaccharide mixture obtained from a cultivation or fermentation process, comprising the following steps 1) broth clarification, 2) ultrafiltration, 3) nanofiltration, 4) mixed bed ion exchange, 5) concentration, and 6) concentrating to a syrup of at least 40% dry matter.

Purification of an oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, and 6) concentrating to a syrup of at least 40% dry matter.

Purification of an oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, and 6) concentrating to a syrup of at least 40% dry matter.

Purification of an oligosaccharide mixture obtained from a cultivation or fermentation process, comprising the following steps 1) broth clarification through ultrafiltration, 2) nanofiltration, 4) mixed bed ion exchange, 5) concentration, and 6) concentrating to a syrup of at least 40% dry matter.

Purification of an oligosaccharide mixture obtained from a cultivation or fermentation process, comprising the following steps 1) broth clarification, 2) ultrafiltration, 3) electrodialysis, 4) mixed bed ion exchange, 5) concentration, and 6) concentrating to a syrup of at least 40% dry matter.

Purification of an oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, and 6) concentrating to a syrup of at least 40% dry matter.

Purification of an oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, and 6) concentrating to a syrup of at least 40% dry matter.

Purification of an oligosaccharide mixture obtained from a cultivation or fermentation process, comprising the following steps 1) broth clarification through ultrafiltration, 2) electrodialysis, 4) mixed bed ion exchange, 5) concentration, and 6) concentrating to a syrup of at least 40% dry matter.

Purification of an oligosaccharide mixture obtained from a cultivation or fermentation process, comprising the following steps 1) broth clarification, 2) ultrafiltration, 3) nanofiltration, 4) cation exchange, 5) anion exchange, 6) concentration, 7) Activated Charcoal treatment, and 8) concentrating to a syrup of at least 40% dry matter.

Purification of an oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, 7) Activated Charcoal treatment, and 8) concentrating to a syrup of at least 40% dry matter.

Purification of an oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, 7) Activated Charcoal treatment, and 8) concentrating to a syrup of at least 40% dry matter.

Purification of an oligosaccharide mixture obtained from a cultivation or fermentation process, comprising the following steps 1) broth clarification through ultrafiltration, 2) nanofiltration, 3) cation exchange, 4) anion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification, 2) ultrafiltration, 3) electrodialysis, 4) cation exchange, 5) anion exchange, 6) concentration, 7) Activated Charcoal treatment, and 8) concentrating to a syrup of at least 40% dry matter.

Purification of an oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, 7) Activated Charcoal treatment, and 8) concentrating to a syrup of at least 40% dry matter.

Purification of an oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, 7) Activated Charcoal treatment, and 8) concentrating to a syrup of at least 40% dry matter.

Purification of an oligosaccharide mixture obtained from a cultivation or fermentation process, comprising the following steps 1) broth clarification through ultrafiltration, 2) electrodialysis, 3) cation exchange, 4) anion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification, 2) ultrafiltration, 3) nanofiltration, 4) mixed bed ion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through ultrafiltration, 2) nanofiltration, 4) mixed bed ion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification, 2) ultrafiltration, 3) electrodialysis, 4) mixed bed ion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through ultrafiltration, 2) electrodialysis, 4) mixed bed ion exchange, 5) concentration, 6) Activated Charcoal treatment, and 7) concentrating to a syrup of at least 40% dry matter.

Purification of an oligosaccharide mixture obtained from a cultivation or fermentation process comprising the following steps 1) broth clarification, 2) ultrafiltration, 3) cation exchange, 4) anion exchange, 5) concentration, 6) Activated Charcoal treatment, and 7) concentrating to a syrup of at least 40% dry matter.

Purification of a neutral oligosaccharide mixture obtained from a cultivation or fermentation process, comprising the following steps 1) broth clarification, 2) ultrafiltration, 3) nanofiltration, 4) cation exchange, 5) anion exchange, 6) concentration, and 7) spray drying.

Purification of a neutral oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, and 7) spray drying.

Purification of a neutral oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, and 7) spray drying.

Purification of a neutral oligosaccharide mixture obtained from a cultivation or fermentation process, comprising the following steps 1) broth clarification through ultrafiltration, 2) nanofiltration, 3) cation exchange, 4) anion exchange, 5) concentration, and 6) spray drying.

Purification of a neutral oligosaccharide mixture obtained from a cultivation or fermentation process, comprising the following steps 1) broth clarification, 2) ultrafiltration, 3) electrodialysis, 4) cation exchange, 5) anion exchange, 6) concentration, and 7) spray drying.

Purification of a neutral oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, and 7) spray drying.

Purification of a neutral oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, and 7) spray drying.

Purification of a neutral oligosaccharide mixture obtained from a cultivation or fermentation process, comprising the following steps 1) broth clarification through ultrafiltration, 2) electrodialysis, 3) cation exchange, 4) anion exchange, 5) concentration, 6) spray drying.

Purification of a neutral oligosaccharide mixture obtained from a cultivation or fermentation process, comprising the following steps 1) broth clarification, 2) ultrafiltration, 3) nanofiltration, 4) mixed bed ion exchange, 5) concentration, and 6) spray drying.

Purification of a neutral oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, and 6) spray drying.

Purification of a neutral oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, and 6) spray drying.

Purification of a neutral oligosaccharide mixture obtained from a cultivation or fermentation process, comprising the following steps 1) broth clarification through ultrafiltration, 2) nanofiltration, 4) mixed bed ion exchange, 5) concentration, and 6) spray drying.

Purification of a neutral oligosaccharide mixture obtained from a cultivation or fermentation process, comprising the following steps 1) broth clarification, 2) ultrafiltration, 3) electrodialysis, 4) mixed bed ion exchange, 5) concentration, and 6) spray drying.

Purification of a neutral oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, and 6) spray drying.

Purification of a neutral oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, and 6) spray drying.

Purification of a neutral oligosaccharide mixture obtained from a cultivation or fermentation process, comprising the following steps 1) broth clarification through ultrafiltration, 2) electrodialysis, 4) mixed bed ion exchange, 5) concentration, and 6) spray drying.

Purification of a neutral oligosaccharide mixture obtained from a cultivation or fermentation process, comprising the following steps 1) broth clarification, 2) ultrafiltration, 3) nanofiltration, 4) cation exchange, 5) anion exchange, 6) concentration, 7) Activated Charcoal treatment, and 8) spray drying.

Purification of a neutral oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, 7) Activated Charcoal treatment, and 8) spray drying.

Purification of a neutral oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, 7) Activated Charcoal treatment, and 8) spray drying.

Purification of a neutral oligosaccharide mixture obtained from a cultivation or fermentation process, comprising the following steps 1) broth clarification through ultrafiltration, 2) nanofiltration, 3) cation exchange, 4) anion exchange, 5) concentration, 6) Activated Charcoal treatment, and 7) spray drying.

Purification of a neutral oligosaccharide mixture obtained from a cultivation or fermentation process, comprising the following steps 1) broth clarification, 2) ultrafiltration, 3) electrodialysis, 4) cation exchange, 5) anion exchange, 6) concentration, 7) Activated Charcoal treatment, and 8) spray drying.

Purification of a neutral oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, 7) Activated Charcoal treatment 8) spray drying.

Purification of a neutral oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, 7) Activated Charcoal treatment, and 8) spray drying.

Purification of a neutral oligosaccharide mixture obtained from a cultivation or fermentation process, comprising the following steps 1) broth clarification through ultrafiltration, 2) electrodialysis, 3) cation exchange, 4) anion exchange, 5) concentration, 6) Activated Charcoal treatment, and 7) spray drying.

Purification of a neutral oligosaccharide mixture obtained from a cultivation or fermentation process, comprising the following steps 1) broth clarification, 2) ultrafiltration, 3) nanofiltration, 4) mixed bed ion exchange, 5) concentration, 6) Activated Charcoal treatment, and 7) spray drying.

Purification of a neutral oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, 6) Activated Charcoal treatment, and 7) spray drying.

Purification of a neutral oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, 6) Activated Charcoal treatment, and 7) spray drying.

Purification of a neutral oligosaccharide mixture obtained from a cultivation or fermentation process, comprising the following steps 1) broth clarification through ultrafiltration, 2) nanofiltration, 4) mixed bed ion exchange, 5) concentration, 6) Activated Charcoal treatment, and 7) spray drying.

Purification of a neutral oligosaccharide mixture obtained from a cultivation or fermentation process, comprising the following steps 1) broth clarification, 2) ultrafiltration, 3) electrodialysis, 4) mixed bed ion exchange, 5) concentration, 6) Activated Charcoal treatment, and 7) spray drying.

Purification of a neutral oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, 6) Activated Charcoal treatment, and 7) spray drying.

Purification of a neutral oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, 6) Activated Charcoal treatment, and 7) spray drying.

Purification of a neutral oligosaccharide mixture obtained from a cultivation or fermentation process, comprising the following steps 1) broth clarification through ultrafiltration, 2) electrodialysis, 4) mixed bed ion exchange, 5) concentration, 6) Activated Charcoal treatment, and 7) spray drying.

Purification of a neutral and charged oligosaccharide mixture obtained from a cultivation or fermentation process, comprising the following steps 1) broth clarification, 2) ultrafiltration, 3) nanofiltration, 4) cation exchange, 5) anion exchange, 6) concentration, and 7) spray drying.

Purification of a neutral and charged oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, and 7) spray drying.

Purification of a neutral and charged oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, and 7) spray drying.

Purification of a neutral and charged oligosaccharide mixture obtained from a cultivation or fermentation process, comprising the following steps 1) broth clarification through ultrafiltration, 2) nanofiltration, 3) cation exchange, 4) anion exchange, 5) concentration, and 6) spray drying.

Purification of a neutral and charged oligosaccharide mixture obtained from a cultivation or fermentation process, comprising the following steps 1) broth clarification, 2) ultrafiltration, 3) electrodialysis, 4) cation exchange, 5) anion exchange, 6) concentration, and 7) spray drying.

Purification of a neutral and charged oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, and 7) spray drying.

Purification of a neutral and charged oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, and 7) spray drying.

Purification of a neutral and charged oligosaccharide mixture obtained from a cultivation or fermentation process, comprising the following steps 1) broth clarification through ultrafiltration, 2) electrodialysis, 3) cation exchange, 4) anion exchange, 5) concentration, and 6) spray drying.

Purification of a neutral and charged oligosaccharide mixture obtained from a cultivation or fermentation process, comprising the following steps 1) broth clarification, 2) ultrafiltration, 3) nanofiltration, 4) mixed bed ion exchange, 5) concentration, and 6) spray drying.

Purification of a neutral and charged oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, and 6) spray drying.

Purification of a neutral and charged oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, and 6) spray drying.

Purification of a neutral and charged oligosaccharide mixture obtained from a cultivation or fermentation process, comprising the following steps 1) broth clarification through ultrafiltration, 2) nanofiltration, 4) mixed bed ion exchange, 5) concentration, and 6) spray drying.

Purification of a neutral and charged oligosaccharide mixture obtained from a cultivation or fermentation process, comprising the following steps 1) broth clarification, 2) ultrafiltration, 3) electrodialysis, 4) mixed bed ion exchange, 5) concentration, and 6) spray drying.

Purification of a neutral and charged oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, and 6) spray drying.

Purification of a neutral and charged oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, and 6) spray drying.

Purification of a neutral and charged oligosaccharide mixture obtained from a cultivation or fermentation process, comprising the following steps 1) broth clarification through ultrafiltration, 2) electrodialysis, 4) mixed bed ion exchange, 5) concentration, 6) spray drying.

Purification of a neutral and charged oligosaccharide mixture obtained from a cultivation or fermentation process, comprising the following steps 1) broth clarification, 2) ultrafiltration, 3) nanofiltration, 4) cation exchange, 5) anion exchange, 6) concentration, 7) Activated Charcoal treatment, and 8) spray drying.

Purification of a neutral and charged oligosaccharide mixture obtained from a cultivation or fermentation process, comprising the following steps 1) broth clarification through microfiltration, 2) ultrafiltration, 3) nanofiltration, 4) cation exchange, 5) anion exchange, 6) concentration, 7) Activated Charcoal treatment, and 8) spray drying.

Purification of a neutral and charged oligosaccharide mixture obtained from a cultivation or fermentation process, comprising the following steps 1) broth clarification through centrifugation, 2) ultrafiltration, 3) nanofiltration, 4) cation exchange, 5) anion exchange, 6) concentration, 7) Activated Charcoal treatment, and 8) spray drying.

Purification of a neutral and charged oligosaccharide mixture obtained from a cultivation or fermentation process, comprising the following steps 1) broth clarification through ultrafiltration, 2) nanofiltration, 3) cation exchange, 4) anion exchange, 5) concentration, 6) Activated Charcoal treatment, and 7) spray drying.

Purification of a neutral and charged oligosaccharide mixture obtained from a cultivation or fermentation process, comprising the following steps 1) broth clarification, 2) ultrafiltration, 3) electrodialysis, 4) cation exchange, 5) anion exchange, 6) concentration, 7) Activated Charcoal treatment, and 8) spray drying.

Purification of a neutral and charged oligosaccharide mixture obtained from a cultivation or fermentation process, comprising the following steps 1) broth clarification through microfiltration, 2) ultrafiltration, 3) electrodialysis, 4) cation exchange, 5) anion exchange, 6) concentration, 7) Activated Charcoal treatment, and 8) spray drying.

Purification of a neutral and charged oligosaccharide mixture obtained from a cultivation or fermentation process, comprising the following steps 1) broth clarification through centrifugation, 2) ultrafiltration, 3) electrodialysis, 4) cation exchange, 5) anion exchange, 6) concentration, 7) Activated Charcoal treatment, and 8) spray drying.

Purification of a neutral and charged oligosaccharide mixture obtained from a cultivation or fermentation process, comprising the following steps 1) broth clarification through ultrafiltration, 2) electrodialysis, 3) cation exchange, 4) anion exchange, 5) concentration, 6) Activated Charcoal treatment, and 7) spray drying.

Purification of a neutral and charged oligosaccharide mixture obtained from a cultivation or fermentation process, comprising the following steps 1) broth clarification, 2) ultrafiltration, 3) nanofiltration, 4) mixed bed ion exchange, 5) concentration, 6) Activated Charcoal treatment, and 7) spray drying.

Purification of a neutral and charged oligosaccharide mixture obtained from a cultivation or fermentation process, comprising the following steps 1) broth clarification through microfiltration, 2) ultrafiltration, 3) nanofiltration, 4) mixed bed ion exchange, 5) concentration, 6) Activated Charcoal treatment, and 7) spray drying.

Purification of a neutral and charged oligosaccharide mixture obtained from a cultivation or fermentation process, comprising the following steps 1) broth clarification through centrifugation, 2) ultrafiltration, 3) nanofiltration, 4) mixed bed ion exchange, 5) concentration, 6) Activated Charcoal treatment, and 7) spray drying.

Purification of a neutral and charged oligosaccharide mixture obtained from a cultivation or fermentation process, comprising the following steps 1) broth clarification through ultrafiltration, 2) nanofiltration, 4) mixed bed ion exchange, 5) concentration, 6) Activated Charcoal treatment, and 7) spray drying.

Purification of a neutral and charged oligosaccharide mixture obtained from a cultivation or fermentation process, comprising the following steps 1) broth clarification, 2) ultrafiltration, 3) electrodialysis, 4) mixed bed ion exchange, 5) concentration, 6) Activated Charcoal treatment, and 7) spray drying.

Purification of a neutral and charged oligosaccharide mixture obtained from a cultivation or fermentation process, comprising the following steps 1) broth clarification through microfiltration, 2) ultrafiltration, 3) electrodialysis, 4) mixed bed ion exchange, 5) concentration, 6) Activated Charcoal treatment, and 7) spray drying.

Purification of a neutral and charged oligosaccharide mixture obtained from a cultivation or fermentation process, comprising the following steps 1) broth clarification through centrifugation, 2) ultrafiltration, 3) electrodialysis, 4) mixed bed ion exchange, 5) concentration, 6) Activated Charcoal treatment, and 7) spray drying.

Purification of a neutral and charged oligosaccharide mixture obtained from a cultivation or fermentation process, comprising the following steps 1) broth clarification through ultrafiltration, 2) electrodialysis, 4) mixed bed ion exchange, 5) concentration, 6) Activated Charcoal treatment, and 7) spray drying.

1.-80. (canceled)

81. A method of producing a purified mixture of different oligosaccharides, the method comprising the steps of:
culturing at least one cell that synthesizes a mixture of different oligosaccharides in a suitable cultivation or fermentation medium to form a cultivation or fermentation broth, and
purifying the oligosaccharide mixture from the cultivation or fermentation broth by clarifying the cultivation or fermentation broth, and
i) removing salts, medium components, or salts and medium components from the clarified cultivation or fermentation broth, and
ii) concentrating the oligosaccharide mixture in the clarified cultivation or fermentation broth, or
iii) a combination of i) and ii)
so as to produce a solution comprising the purified mixture of oligosaccharides.

82. The method according to claim 81, wherein step iii) precedes step ii).

83. The method according to claim 81, wherein the oligosaccharide mixture comprises at least one neutral and at least one charged oligosaccharide.

84. The method according to claim 81, wherein the at least one cell is cultured in a minimal salt medium with a carbon source on which the at least one cell grows.

85. The method according to claim 81,
wherein the mixture of oligosaccharides' combined purity in the fermentation broth is less than 80% on total dry solid, or
wherein the combined purity of the purified mixture of oligosaccharides in the solution is equal to or more than 80% on total dry solid.

86. The method according to claim 81, wherein step i) comprises one or more of clarification, clearing, filtration, microfiltration, centrifugation, decantation, and ultrafiltration.

87. The method according to claim 86, wherein step i) comprises subjecting the cultivation or fermentation broth to two membrane filtration steps using different membranes.

88. The method according to claim 81, wherein step ii) comprises at least one or more of nanofiltration, dialysis, electrodialysis, use of activated charcoal or carbon, use of solvents, use of alcohols, use of aqueous alcohol mixtures, use of charcoal, tangential flow high-performance filtration, tangential flow ultrafiltration, affinity chromatography, ion exchange, cation exchange, anion exchange, mixed bed ion exchange, simulated moving bed chromatography, ion exchange chromatography, hydrophobic interaction chromatography, gel filtration, ligand exchange chromatography, column chromatography, cation exchange adsorbent resin, or use of ion exchange resin.

89. The method according to claim 88, wherein step ii) comprises anion exchange wherein the anion exchange resin has a moisture content of 30-48%.

90. The method according to claim 81, wherein step ii) comprises treatment with a mixed bed ion exchange resin.

91. The method according to claim 81, wherein the mixture of oligosaccharides comprises at least one mammalian milk oligosaccharide.

92. The method according to claim 81, wherein step i) comprises a first step of clarification by microfiltration.

93. The method according to claim 81, wherein step i) comprises a first step of clarification by centrifugation.

94. The method according to claim 81, wherein step i) comprises a first step of clarification by flocculation.

95. The method according to claim 81, wherein step i) comprises ultrafiltration.

96. The method according to claim 81, wherein step ii) comprises nanofiltration, electrodialysis, or a combination of nanofiltration and electrodialysis.

97. The method according to claim 95, wherein the ultrafiltration permeate of step i) is nanofiltered, electrodialysed, or nanofiltered and electrodialysed in step ii).

98. The method according to claim 81, wherein step i) is ultrafiltration, and step ii) is nanofiltration, electrodialysis, or nanofiltration and electrodialysis combined with treatment with an ion exchange resin or chromatography.

99. The method according to claim 81, wherein step ii) comprises electrodialysis.

100. The method according to claim 97, wherein the molecular weight cut-off of the nanofiltration membrane in step ii) is lower than that of the ultrafiltration membrane in step i).

101. The method according to claim 81, wherein step i) is preceded by an enzymatic treatment.

102. The method according to claim 81, wherein the purified oligosaccharide mixture solution is dried.

103. The method according to claim 102, wherein the purified oligosaccharide mixture solution is dried by spray-drying or freeze-drying the purified oligosaccharide mixture solution and wherein the pH of the solution is lower than 5.0.

104. A dried purified mixture of oligosaccharides powder produced by the method according to claim 102, wherein the powder exhibits:
a loose bulk density of from about 500 g/L to about 700 g/L,
a 100× tapped bulk density of from about 600 g/L to about 850 g/L,
a 625× tapped bulk density of from about 600 g/L to about 900 g/L, or
a 1250× tapped bulk density of from about 650 g/L to about 900 g/L.

105. A method for producing a purified charged oligosaccharide, the method comprising the following steps:
culturing at least one cell that synthesizes a charged oligosaccharide in a suitable cultivation or fermentation medium to form a cultivation or fermentation broth, and
purifying the charged oligosaccharide from the cultivation or fermentation broth by clarifying the cultivation or fermentation broth, and
i) removing salts, medium components, or salts and medium components from the clarified cultivation or fermentation broth, and
ii) concentrating the oligosaccharide mixture in the clarified cultivation or fermentation broth, or
iii) a combination of i) and ii),
wherein step ii) comprises treatment with a) a mixed bed ion exchange, or b) cation and anion exchange, thereby providing a solution comprising the purified charged oligosaccharide.

106. The method according to claim 105, wherein the anion exchange resin used in a) or b) has a moisture content of 30-48%.

107. The method according to claim 105, wherein the treatment is a) a mixed bed ion exchange and the mixed bed ion exchange resin is a mixed bed column of cross-linked polystyrene-divinylbenzene anion exchange resin and cross-linked polystyrene-sulfonic acid cation exchange resin mixed in a ratio 1.1:1 to 1.9:1.
